(19) United States
(12) Patent Application Publication
(10) Pub. No.: US 2009/0311285 A1
(43) Pub. Date: Dec. 17, 2009

(54) VACCINE

(75) Inventors: Ralph Leon Biemans, Rixensart (BE); Pierre Duvivier, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GlaxoSmith Kline Biologicals s.a. a corporation

(21) Appl. No.: 12/521,969

(22) PCT Filed: Jan. 2, 2008
(86) PCT No.: PCT/EP2008/050021

§ 371 Date: Jul. 1, 2009

(30) Foreign Application Priority Data
Jan. 4, 2007 (GB) 0700135.7

(51) Int. Cl. A61K 39/385; A61P 31/20; A61P 31/14; A61P 33/06; A61P 31/04; A61P 31/16

(52) U.S. Cl. 424/194.1

(57) ABSTRACT

The present invention relates to immunogenic compositions or vaccines comprising a Vi capsular saccharide-protein carrier conjugate, methods of making the immunogenic compositions or vaccines and uses thereof.

The present invention relates to improved methods of conducting carbodiimide condensation reactions. In particular, it relates to the conjugation of saccharides and proteins using carbodiimide condensation. It also relates to immunogenic compositions that may be made comprising the saccharide-protein conjugates of the invention.

The use of bacterial capsular polysaccharides has been widely used in immunology for many years for the prevention of bacterial disease. A problem with such a use, however, is the T-independent nature of the immune response. These antigens are thus poorly immunogenic in young children. This problem has been overcome through conjugating the polysaccharide antigens to a protein carrier (a source of T-helper epitopes) which may then by used to elicit a T-dependent immune response, even in the first year of life.

Various conjugation techniques are known in the art. Conjugates can be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508. The conjugation method may alternatively rely on activation of hydroxyl groups of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the cyanate ester can be coupled with hexane diamine or adipic acid dihydrazide (ADH or AH) and the amino-derivatised saccharide is conjugated to the carrier protein using carbodiimide (e.g. EDAC or EDC) chemistry via a carboxyl group on the protein carrier. Such conjugates are described in PCT published application WO 93/15760 Uniformed Services University and WO 95/08348 and WO 96/29094. See also Chu C. et al Infect. Immunity, 1983 245 256.

In general the following types of chemical groups on a protein carrier can be used for coupling/conjugation:

A) Carboxyl (for instance via aspartic acid or glutamic acid) which may be conjugated to natural or derivatised amino groups on saccharide moieties using carbodiimide chemistry;
B) Amino group (for instance via lysine) which may be conjugated to natural or derivatised carboxyl groups on saccharide moieties using carbodiimide chemistry;
C) Sulphydryl (for instance via cysteine);
D) Hydroxyl group (for instance via tyrosine);
E) Imidazolyl group (for instance via histidine);
F) Guanidyl group (for instance via arginine); and
G) Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

### Direct Coupling Approaches:

Saccharide-OH+CNBr or CDAP - - - >cyanate ester+NH2-Prot - - - >conjugate
Saccharide-aldehyde+NH2-Prot - - - >Schiff base+NaCNBH3 - - - >conjugate
Saccharide-COOH+NH2-Prot+EDAC - - - >conjugate
Saccharide-NH2+COOH-Prot+EDAC - - - >conjugate

### Indirect Coupling Via Spacer (Linker) Approaches:

Saccharide-OH+CNBr or CDAP - - - >cyanate ester+NH2 - - - NH2 - - - >saccharide - - - NH2+COOH-Prot+EDAC - - - >conjugate
Saccharide-OH+CNBr or CDAP - - - >cyanate ester+NH2 - - - SH - - - >saccharide - - - SH+SH-Prot (native Protein with an exposed cysteine or obtained after modification of amino groups of the protein by SPDP for instance) - - - >saccharide-S—S-Prot
Saccharide-OH+CNBr or CDAP - - - >cyanate ester+NH2 - - - SH - - - >saccharide - - - SH+maleimide-Prot (modification of amino groups) - - - >conjugate
Saccharide-OH+CNBr or CDAP - - - >cyanate ester+NH2- - - SH - - - >Saccharide-SH+haloacetylated-Prot - - - >Conjugate
Saccharide-COOH+EDAC+NH2 - - - NH2 - - - >saccharide - - - NH2+EDAC+COOH-Prot - - - >conjugate
Saccharide-COOH+EDAC+NH2- - - SH - - - >saccharide - - - SH+SH-Prot (native Protein with an exposed cysteine or obtained after modification of amino groups of the protein by SPDP for instance) - - - >saccharide-S—S-Prot
Saccharide-COOH+EDAC+NH2- - - SH - - - >saccharide - - - SH+maleimide-Prot (modification of amino groups) - - - >conjugate
Saccharide-COOH+EDAC+NH2 - - - SH - - - >Saccharide-SH+haloacetylated-Prot - - - >Conjugate
Saccharide-Aldehyde+NH2 - - - NH2 - - - >saccharide - - - NH2+EDAC+COOH-Prot - - - >conjugate

Note, where EDAC is described herein, any suitable carbodiimide may instead be used.

As can be observed carbodiimide chemistry (e.g. using EDAC) is very convenient for conjugation reactions as it makes use of groups on the saccharide and/or protein which may be naturally present or easily inserted by derivatisation. It also conveniently links moieties through a peptide bond.

Carbodiimides (RN═C═NR′) are unsaturated compounds with an allene structure (Nakajima and Ikada 1995 Bioconjugate Chem. 6:123-130; Hoare and Koshland 1967 JBC 242:2447-2453). The chemical is relatively unstable at its reaction pH (4.5-6.5), and therefore all components of the saccharide/protein/carbodiimide conjugation reaction tend to be added together in the art.

The present inventors have found that depending on the nature of the saccharide and protein to be conjugated, better characteristics of the final conjugate for vaccine use may be achieved by adding a certain component of the reaction slowly to the mix. In so doing one or more benefits/improvements may be realised such as: saccharide yield in the conjugate, sterile filterability of the conjugate, better control of the conjugation, easier reproducibility, and/or prevention of intra-moiety cross-links.

Accordingly, in one embodiment there is provided a method of conjugating a saccharide to a protein carrier using carbodiimide condensation chemistry, wherein the saccharide comprises (for instance as part of its repeating unit), or has been derivatised to comprise, amino and/or carboxyl groups, and wherein the protein carrier comprises, or has been derivatised to comprise, amino and/or carboxyl groups, comprising the steps of:
I)—if the protein carrier comprises both amino and carboxyl groups and the saccharide comprises either amino or carboxyl groups:
a) mixing the saccharide and aliquot of carbodiimide required to perform the conjugation, and
b) adding the aliquot of protein carrier required over a period of 35 seconds to 6 hours;
II)—if the saccharide comprises both amino and carboxyl groups and the protein carrier comprises either amino or carboxyl groups:
a) mixing the protein carrier and aliquot of carbodiimide required to perform the conjugation, and
b) adding the aliquot of saccharide required over a period of 35 seconds to 6 hours;
III)—if the saccharide comprises both amino and carboxyl groups and the protein carrier comprises both amino and carboxyl groups:
a) mixing the protein carrier and saccharide, and
b) adding the aliquot of carbodiimide required to perform the conjugation over a period of 35 seconds to 6 hours.

### DETAILED DESCRIPTION

Any suitable carbodiimide may be used as long as it is capable of conjugating saccharides and proteins in an aqueous medium. In one embodiment the carbodiimide may be EDAC (1-ethyl-3-(3-dimethyl-aminopropyl) carbodiimide) [also known as EDC] or it may be a carbodiimide other than EDAC. Where EDAC or EDC is mentioned herein in any embodiment, it is envisioned that any carbodiimide may alternatively be used.

The term “saccharide” throughout this specification may indicate polysaccharide or oligosaccharide and includes both. It may indicate lipopolysaccharide (LPS) or lipooliogosaccharide (LOS). Before use Polysaccharides (such as bacterial polysaccharides) may be isolated from a source strain (e.g. of bacteria) or isolated from the source strain and sized to some degree by known methods (see for example EP497524 and EP497525; Shousun Chen Szu et al.—Carbohydrate Research Vol 152 p 7-20 (1986)) for instance by microfluidisation. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides.

The term “protein carrier” is intended to cover both small peptides and large polypeptides (>10 kDa). Clearly large polypeptides are more likely to contain both reactive amino and carboxyl groups without any modification.

For the purposes of the invention, “native polysaccharide” refers to a saccharide that has not been subjected to a process, the purpose of which is to reduce the size of the saccharide. A polysaccharide can become slightly reduced in size during normal purification procedures. Such a saccharide is still native. Only if the polysaccharide has been subjected to sizing techniques would the polysaccharide not be considered native.

For the purposes of the invention, “sized by a factor up to x2” means that the saccharide is subject to a process intended to reduce the size of the saccharide but to retain a size more than half the size of the native polysaccharide. X3, x4 etc. are to be interpreted in the same way i.e. the saccharide is subject to a process intended to reduce the size of the polysaccharide but to retain a size more than a third, a quarter etc. the size of the native polysaccharide.

The 35 second to 6 hour time period in step b) of the method for the addition of the full aliquot of the final component can be 50 seconds to 5 hours, 1 minute to 4 hours, 2 minutes to 3 hours, 3 minutes to 2 hours, 4 to 60 minutes, 5 to 50 minutes, 6 to 40 minutes, 7 to 30 minutes or 8 to 20 minutes. It may be 1 minute to 5 hours, 10 minutes to 4 hours, 20 minutes to 3 hours, 30 minutes to 2 hours, 40 to 90 minutes, or 50 to 70 minutes. This time can be adjusted according to the precise saccharide and protein being conjugated.

In one embodiment the aliquot of the final component (e.g. of carbodiimide, saccharide or protein) is added to the reaction mixture at a constant rate during the time period (this is conveniently achieved using a pump operating at a constant rate). Alternatively it may be added in stages over the time period. Although this may be done in many ways, in general parts of the aliquot should be added throughout the period. For instance at least one quarter of the aliquot may be added over the first half of the period, and at least one quarter of the aliquot over the second half of the period. The total amount of the aliquot ‘a’ measured, for instance, in mL or mg may be added in 4-100 stages (‘s’) throughout the period. In one embodiment the stages are arranged such that an even amount (a/s) is introduced at all the stages. In one embodiment the stages are evenly spaced throughout the period ‘p’ (in seconds). Thus if one stage takes place at time zero of the period ‘p’, then each subsequent stage could take place at a time which is p/(s−1). The volume of the aliquot of the final component added in step b) may be adjusted in terms of ease of addition of the aliquot to the reaction within the desired time period. The carbodiimide may be added as an aqueous solution (typically buffered at pH 7.5 before being added to the reaction) or as solid powder (EDAC for instance is highly soluble in aqueous media). Of course if the carbodiimide is the last component added to the reaction (situation III step b)), a slow dissolving carbodiimide may be used such that the entire aliquot of powder is added to the reaction all at once but it dissolves at a rate consistent with the desired period over which the aliquot is to be made available to the reaction.

If the protein and/or saccharide has no amino or carboxyl groups (or only has one of these), it may be derivatised to give it one (or to give it the other it does not already have). For instance for a saccharide only comprising reactive hydroxyl groups (e.g. meningococcal serogroup A capsular saccharide), such a group should be used for derivatising on amino or carboxyl groups so that EDAC condensation may be carried out. This may take place within a repeat subunit, or may be a group only present at the end of the saccharide molecule.

It should be noted that where derivatisation takes place, it can be beneficial to only partially derivatise the moiety. For saccharides with repeating subunits, the target epitope may be present in each repeat. Therefore if partial derivatisation takes place (for this it is meant, for example, 0.5-20, 1-15, 3-12, or 5-10% of the targeted reactive group is actually derivatised) this can have the benefit of conserving the majority of the epitopes, and preventing too much cross-linking.

If a saccharide or protein already has amino or carboxyl groups only (e.g. Vi saccharide from Salmonella typhi which naturally has carboxyl but not amino groups), derivatisation can take place to give it the other type of group (i.e. amino groups for Vi). It should be noted, however, that as derivatisation can be partial this action can change the preferred reaction of the invention from a type I to a type III. For instance if Vi saccharide is conjugated to a protein carrier comprising both amino and carboxyl groups situation I adds the aliquot of protein slowly in step b). If the Vi saccharide carboxyl group is partially derivatised with amino groups it will have both carboxyl and amino groups, thus situation III adding the aliquot of carbodiimide slowly in step b) becomes most relevant.

Derivatisation may occur through the addition of a hetero- or homo-bifunctional linker. It may take place with similar chemistry as described above for saccharide-protein conjugation step (e.g. CDAP or carbodiimide chemistry). The linker may have between 4 and 20, 4 and 12, or 5 and 10 carbon atoms. It may have two reactive amino groups, two reactive carboxyl groups, or one of each (e.g. hexane diamine, 6-aminocaproic acid, or adipic acid dihydrazide). Typically derivatization takes place through reacting a large excess of the linker with the saccharide and/or protein carrier to be derivatised. This allows derivatization to take place with minimal intra-moiety cross-linking (which otherwise might be possible if for instance a carboxyl group on a saccharide was being derivatised with amino groups using carbodiimide condensation). Excess linker is readily removed using techniques such as diafiltration.

In one embodiment the saccharide comprises a reactive hydroxyl group as part of its repeating unit which is partially derivatised via an amino group on the linker (e.g. with CDAP chemistry). In another embodiment the saccharide comprises a reactive amino group as part of its repeating unit which is partially derivatised via a carboxyl group on the linker (e.g. with carbodiimide chemistry). In a further embodiment the saccharide comprises a reactive carboxyl group as part of its repeating unit which is partially derivatised via an amino group on the linker (e.g. with carbodiimide chemistry—for instance wherein the carbodiimide in the partial derivatisation step is present at 0.01-0.5, 0.015-0.1, 0.02-0.075, or 0.025-0.05 mg carbodiimide/mg saccharide).

The aliquot of carbodiimide required to perform the conjugation (whether present in step a) or b) of the reaction of the invention) is 0.01 to 3, 0.05 to 2, or 0.09 to 1 mg carbodiimide/mg saccharide (for instance 0.07 to 0.25, or 0.1 to 0.2 mg/mg saccharide). Although these numbers (and quantities of carbodiimide recited herein) are calculated in respect of EDAC being the carbodiimide, these numbers may optionally be adjusted if any other carbodiimide is used by multiplying the numbers in the range by: (molecular weight of other carbodiimide)/(molecular weight of EDAC).

In general, the saccharide may be present in the methods of the invention at a final concentration of 0.5-50 mg/ml in step b). This will depend on the size and nature of the saccharide, and the extent of any derivatisation. For instance for oligosaccharides a larger concentration will be required, but for large polysaccharides a much smaller concentration will be more appropriate. If it is towards the high end of partially derivatised with amino or carboxyl groups a smaller concentration may be appropriate to reduce the possibility of any cross-linking. The protein carrier may be present at a final concentration of 1-50 mg/ml in step b).

The initial ratio of protein carrier to saccharide in the methods of the invention can be 5:1 to 1:5, 4:1 to 1:1, or 3:1 to 2:1 (w/w). Again this will depend on the size and nature of the saccharide, and the extent of any derivatisation.

Salt conditions (e.g. NaCl) may also be varied according to the nature of the saccharide/protein. Usually around 0.2M NaCl may be present in step b) of the methods of the invention, but may be 0-2, 0.1-1 or 0.2-0.5 M.

In terms of pH in step b) of the methods of the invention, the reaction pH may be any pH where the carbodiimide is activated—for instance pH 4.5-6.5, 4.7-6.0, or 5-5.5. This pH is typically maintained throughout the reaction by addition of acid/base as required. EDAC is usually stable at pH 7.5, though if the conjugation requires to be done at higher pH compounds which are known to keep the reaction intermediate stable (such as N-hydroxysuccinimide) may also be present in the reaction in step b), in which case the reaction pH in step b) may be maintained at pH 4.5-7.5.

The reaction temperature during step b) of the methods of the invention can be 4-37, 10-32, 17-30, or 22-27° C., and is typically maintained throughout the reaction.

In the methods of the invention, once the entire aliquot has been added in step b) the reaction is typically maintained for a further 10 minutes to 72 hours, 20 minutes to 48 hours, 30 minutes to 24 hours, 40 minutes to 12 hours, 50 minutes to 6 hours, or 1-3 hours, for instance 10-120, 10-80, 10-50, 20-40, or 25-30 minutes. Once the reaction is completed the pH is adjusted to 7.5-9 (towards the higher end of this if N-hydroxysuccinimide is present) to go back to the stable pH range of carbodiimide.

Once conjugated, the saccharide-protein conjugate may be purified from: unreacted components, free saccharide, etc by injecting it on a size exclusion chromatography column (for instance Sephacryl S400HR, Pharmacia). This is typically carried out at 2-8° C. The conjugate may be sterile filtered then stored. Ultimately an effective dose (for instance 1-20, 2-15, or 3-10 μg saccharide/dose) of the saccharide-protein conjugate can be formulated with a pharmaceutically acceptable excipient (for instance a salt or adjuvant) to manufacture an immunogenic composition or vaccine.

In terms of the saccharides of the invention, any saccharide of viral, fungal, bacterial or eukaryotic source may be conjugated using the methods of the invention. It may be the Vi saccharide from Salmonella typhi, or a saccharide other than Vi. It may be the capsular saccharide Hib from H. influenzae type b, or may be a saccharide other than Hib. In one embodiment the saccharide is a bacterial capsular saccharide, for instance derived from a bacterium selected from a list consisting of: N. meningitidis serogroup A (MenA), B (MenB), C (MenC), W135 (MenW) or Y (MenY), Streptococcus pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F or 33F, Group B Streptococcus group Ia, Ib, II, III, IV, V, VI, or VII, Staphylococcus aureus type 5, Staphylococcus aureus type 8, Salmonella typhi (Vi saccharide), Vibrio cholerae, or H. influenzae type b.

The weight-average molecular weight of the saccharide may be 1000-2000000, 5000-1000000, 10000-500000, 50000-400000, 75000-300000, or 100000-200000. The molecular weight or average molecular weight of a saccharide herein refers to the weight-average molecular weight (Mw) of the saccharide measured prior to conjugation and is measured by MALLS. The MALLS technique is well known in the art and is typically carried out as described in example 2. For MALLS analysis of saccharides, two columns (TSKG6000 and 5000PWxl) may be used in combination and the saccharides are eluted in water. Saccharides are detected using a light scattering detector (for instance Wyatt Dawn DSP equipped with a 10 mW argon laser at 488 nm) and an inferometric refractometer (for instance Wyatt Otilab DSP equipped with a P100 cell and a red filter at 498 nm). In an embodiment, the polydispersity of the saccharide is 1-1.5, 1-1.3, 1-1.2, 1-1.1 or 1-1.05 and after conjugation to a carrier protein, the polydispersity of the conjugate is 1.0-2.5, 1.0-2.0. 1.0-1.5, 1.0-1.2, 1.5-2.5, 1.7-2.2 or 1.5-2.0. All polydispersity measurements are by MALLS.

The saccharide may be either a native polysaccharide or may have been sized by a factor of no more than 2, 4, 6, 8, 10 or 20 fold (for instance by microfluidization [e.g. by Emulsiflex C-50 apparatus] or other known technique [for instance heat, chemical, oxidation, sonication methods]). Oligosaccharides may have been sized substantially further [for instance by known heat, chemical, or oxidation methods].

The structures of most of these saccharides are known (and therefore whether they naturally have any amino or carboxyl groups for carbodiimide chemistry, or any other reactive group which may be derivatised with amino or carboxyl groups (see table below).

| | Natural | Natural | Other reactive |
|---|---|---|---|
| | NH2 group | COOH group | group |
| S. aureus | | | |
| PS5 | No | Yes | OH |
| PS8 | No | Yes | OH |
| N. meningitidis | | | |
| MenA | No | No | OH |
| MenC | No | Yes | OH |
| MenW135 | No | Yes | OH |
| MenY | No | Yes | OH |
| MenB | No (can be | Yes | OH/N-propyl |
| | generated if | | |
| | de-N-acetylated) | | |
| Gp. B Streptococcus | | | |
| Ia, Ib | No | Yes | OH |
| II | No | Yes | OH |
| III | No | Yes | OH |
| IV | No | Yes | OH |
| V | No | Yes | OH |
| VI | No | Yes | OH |
| VII | No | Yes | OH |
| S. typhi | | | |
| Vi | No | Yes | No |
| S. pneumoniae | | | |
| PS1 | Yes | Yes | OH |
| PS3, 4, 5, 8, 9, 12F | No | Yes | OH |
| Vibrio cholorea | | | |
| Capsular saccharide | yes | No | OH |
| H. influenzae B Hib | No | No | OH |
| LOS | | | |
| Nmen/Mcat/Hi | Yes on PEA | Yes on KDO | OH |

The saccharide may be a bacterial lipooligosaccharide or lipopolysaccharide (see above table), for instance derived from a bacterium selected from a list consisting of: N. meningitidis, H. influenzae, E. coli, Salmonella or M. catarrhalis. The LOS may be meningococcal immunotype L2, L3 or L10. It may be detoxified by alkaline treatment of its Lipid A moiety.

In an embodiment, the MenA capsular saccharide, is at least partially O-acetylated such that at least 50%, 60%, 70%, 80%, 90%, 95% or 98% of the repeat units are O-acetylated at least one position. O-acetylation is for example present at least at the O-3 position of at least 50%, 60%, 70%, 80%, 90%, 95% or 98% of the repeat units. In an embodiment, the MenC capsular saccharide, is at least partially O-acetylated such that at least 30%, 40%, 50%, 60%, 70%, 80%, 90%, 95% or 98% of (α2→9)-linked NeuNAc repeat units are O-acetylated at least one or two positions. O-acetylation is for example present at the O-7 and/or O-8 position of at least 30%. 40%, 50%, 60%, 70%, 80%, 90%, 95% or 98% of the repeat units. In an embodiment, the MenW capsular saccharide, is at least partially O-acetylated such that at least 30%, 40%, 50%, 60%, 70%, 80%, 90%, 95% or 98% of the repeat units are O-acetylated at least one or two positions. O-acetylation is for example present at the O-7 and/or O-9 position of at least 30%. 40%, 50%, 60%, 70%, 80%, 90%, 95% or 98% of the repeat units. In an embodiment, the MenY capsular saccharide, is at least partially O-acetylated such that at least 20%, 30%, 40%, 50%, 60%, 70%, 80%, 90%, 95% or 98% of the repeat units are O-acetylated at least one or two positions. O-acetylation is present at the 7 and/or 9 position of at least 20%, 30%, 40%, 50%, 60%, 70%, 80%, 90%, 95% or 98% of the repeat units. The percentage of O-acetylation refers to the percentage of the repeat units containing O-acetylation. This may be measured in the saccharide prior to conjugate and/or after conjugation.

The protein carrier may be any peptide or protein. It may comprise one or more T-helper epitopes. In one embodiment of the invention the protein carrier is selected from the group consisting of: TT, DT, CRM197, fragment C of TT, protein D of H. influenzae, pneumococcal PhtD, and pneumococcal Pneumolysin. The carrier protein may be tetanus toxoid (TT), tetanus toxoid fragment C, non-toxic mutants of tetanus toxin [note all such variants of TT are considered to be the same type of carrier protein for the purposes of this invention], diphtheria toxoid (DT), CRM197, other non-toxic mutants of diphtheria toxin [such as CRM176, CRM 197, CRM228, CRM 45 (Uchida et al J. Biol. Chem. 218; 3838-3844, 1973); CRM 9, CRM 45, CRM102, CRM 103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711] (note all such variants of DT are considered to be the same type of carrier protein for the purposes of this invention), pneumococcal pneumolysin (Kuo et al (1995) Infect Immun 63; 2706-13), OMPC (meningococcal outer membrane protein—usually extracted from N. meningitidis serogroup B—EP0372501), synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al (2001) Eur J Immunol 31; 3816-3824) such as N19 protein (Baraldoi et al (2004) Infect Immun 72; 4884-7) pneumococcal surface protein PspA (WO 02/091998), iron uptake proteins (WO 01/72337), toxin A or B of C. difficile (WO 00/61761), H. influenzae Protein D (EP594610 and WO 00/56360), pneumococcal PhtA (WO 98/18930, also referred to Sp36), pneumococcal PhtD (disclosed in WO 00/37105, and is also referred to Sp036D), pneumococcal PhtB (disclosed in WO 00/37105, and is also referred to Sp036B), or PhtE (disclosed in WO00/30299 and is referred to as BVH-3).

In a further aspect of the invention there is provided a saccharide-protein carrier conjugate (or an immunogenic composition or vaccine) obtainable or obtained by the method of the invention. Thus the methods of the invention may be incorporated within a method of making an immunogenic composition or vaccine of the invention through carrying out the conjugation method of the invention and formulating the resulting saccharide-protein carrier conjugate in an immunogenic composition or vaccine (for example by formulating the conjugate with a pharmaceutically acceptable excipient).

A use of the immunogenic composition or vaccine of the invention in the manufacture of a medicament for the prevention or treatment of disease, and a method of preventing or treating disease comprising the step of administering an effective dose of the immunogenic composition or vaccine of the invention to a patient in need thereof is further provided. The use or method may be such that the disease is caused by a bacterium selected from a list consisting of: N. meningitidis, Streptococcus pneumoniae, M. catarrhalis, Group B Streptococcus, Staphylococcus aureus, Salmonella typhi, Vibrio cholerae, E. coli, and H. influenzae.

The immunogenic compositions of the invention may also comprise a DTPa or DTPw vaccine (for instance one containing DT, TT, and either a whole cell pertussis (Pw) vaccine or an acellular pertussis (Pa) vaccine (comprising for instance pertussis toxoid, FHA, pertactin, and, optionally agglutinogens 2 and 3). Such combinations may also comprise a vaccine against hepatitis B (for instance it may comprise hepatitis B surface antigen [HepB], optionally adsorbed onto aluminium phosphate). In one embodiment the immunogenic composition of the invention comprises Hib, MenA and MenC saccharide conjugates, or Hib and MenC saccharide conjugates, or Hib, MenC and MenY saccharide conjugates, or MenA, MenC, MenW and MenY saccharide conjugates, wherein at least one, two or all the saccharide conjugates are made according the method of the invention.

Immunogenic compositions of the invention optionally comprise additional viral antigens conferring protection against disease caused by measles and/or mumps and/or rubella and/or varicella. For example, immunogenic composition of the invention contains antigens from measles, mumps and rubella (MMR) or measles, mumps, rubella and varicella (MMRV). In an embodiment, these viral antigens are optionally present in the same container as the meningococcal and/or Hib saccharide conjugate(s) present in the composition. In an embodiment, these viral antigens are lyophilised.

In an embodiment, the immunogenic composition of the invention further comprises an antigen from N. meningitidis serogroup B. The antigen is optionally an outer membrane vesicle preparation from N. meningitidis serogroup B as described in EP301992, WO 01/09350, WO 04/14417, WO 04/14418 and WO 04/14419.

In general, the immunogenic composition of the invention may comprise a dose of each saccharide conjugate between 0.1 and 20 μg, 2 and 10 μg, 2 and 6 μg or 4 and 7 μg of saccharide.

“Around” or “approximately” are defined as within 10% more or less of the given FIGURE for the purposes of the invention.

In an embodiment, the immunogenic composition of the invention is adjusted to or buffered at, or adjusted to between pH 7.0 and 8.0, pH 7.2 and 7.6 or around or exactly pH 7.4.

The immunogenic composition or vaccines of the invention are optionally lyophilised in the presence of a stabilising agent for example a polyol such as sucrose or trehalose.

Optionally, the immunogenic composition or vaccine of the invention contains an amount of an adjuvant sufficient to enhance the immune response to the immunogen. Suitable adjuvants include, but are not limited to, aluminium salts (aluminium phosphate or aluminium hydroxide), squalene mixtures (SAF-1), muramyl peptide, saponin derivatives, mycobacterium cell wall preparations, monophosphoryl lipid A, mycolic acid derivatives, non-ionic block copolymer surfactants, Quil A, cholera toxin B subunit, polyphosphazene and derivatives, and immunostimulating complexes (ISCOMs) such as those described by Takahashi et al. (1990) Nature 344:873-875.

For N. meningitidis or HibMen combinations, it may be advantageous not to use any aluminium salt adjuvant or any adjuvant at all.

As with all immunogenic compositions or vaccines, the immunologically effective amounts of the immunogens must be determined empirically. Factors to be considered include the immunogenicity, whether or not the immunogen will be complexed with or covalently attached to an adjuvant or carrier protein or other carrier, route of administrations and the number of immunising dosages to be administered.

The active agent can be present in varying concentrations in the pharmaceutical composition or vaccine of the invention. Typically, the minimum concentration of the substance is an amount necessary to achieve its intended use, while the maximum concentration is the maximum amount that will remain in solution or homogeneously suspended within the initial mixture. For instance, the minimum amount of a therapeutic agent is optionally one which will provide a single therapeutically effective dosage. For bioactive substances, the minimum concentration is an amount necessary for bioactivity upon reconstitution and the maximum concentration is at the point at which a homogeneous suspension cannot be maintained. In the case of single-dosed units, the amount is that of a single therapeutic application. Generally, it is expected that each dose will comprise 1-100 μg of protein antigen, optionally 5-50 μg or 5-25 μg. For example, doses of bacterial saccharides are 10-20 μg, 5-10 μg, 2.5-5 μg or 1-2.5 μg of saccharide in the conjugate.

The vaccine preparations of the present invention may be used to protect or treat a mammal (for example a human patient) susceptible to infection, by means of administering said vaccine via systemic or mucosal route. A human patient is optionally an infant (under 12 months), a toddler (12-24, 12-16 or 12-14 months), a child (2-10, 3-8 or 3-5 years) an adolescent (12-21, 14-20 or 15-19 years) or an adult. These administrations may include injection via the intramuscular, intraperitoneal, intradermal or subcutaneous routes; or via mucosal administration to the oral/alimentary, respiratory, genitourinary tracts. Intranasal administration of vaccines for the treatment of pneumonia or otitis media is preferred (as nasopharyngeal carriage of pneumococci can be more effectively prevented, thus attenuating infection at its earliest stage). Although the vaccine of the invention may be administered as a single dose, components thereof may also be co-administered together at the same time or at different times (for instance if saccharides are present in a vaccine these could be administered separately at the same time or 1-2 weeks after the administration of a bacterial protein vaccine for optimal coordination of the immune responses with respect to each other). In addition to a single route of administration, 2 different routes of administration may be used. For example, viral antigens may be administered ID (intradermal), whilst bacterial proteins may be administered IM (intramuscular) or IN (intranasal). If saccharides are present, they may be administered IM (or ID) and bacterial proteins may be administered IN (or ID). In addition, the vaccines of the invention may be administered IM for priming doses and IN for booster doses.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

A further aspect of the invention is a process for making the immunogenic composition or vaccine of the invention, comprising the step of mixing the MenA and MenC saccharides of the invention made by the method of the invention, with MenW and MenY that have not been made according to the invention, and with a pharmaceutically acceptable excipient.

Salmonella typhi Immunogenic Compositions/Vaccines of the Invention

In one embodiment the immunogenic composition or vaccine of the invention comprises a Vi saccharide-protein carrier conjugate made according to the processes of the invention and a pharmaceutically acceptable excipient. The Vi saccharide-protein carrier conjugate may comprise 0.5-15, 1-10, 2.0-7.5 or 2.5-5 μg of Vi saccharide per human dose.

The Vi saccharide from Salmonella typhi in the conjugate may be the same as that in the registered product Typherix® (GlaxoSmithKline Biologicals s.a.), described in EP1107787. In one embodiment, the Vi saccharide conjugates of the invention may be adsorbed onto an aluminium salt such as aluminium hydroxide, or aluminium phosphate, or a mixture of both aluminium hydroxide and aluminium phosphate. In one embodiment the Vi saccharide conjugate may be unadsorbed onto an adjuvant, e.g. an aluminium adjuvant salt.

In one aspect the immunogenic composition or vaccine of the invention further comprises a Hib capsular saccharide-protein carrier conjugate. This may be made according to the process of the invention, or by any method known in the art. For example it may be the Hiberix® product of GlaxoSmithKline Biologicals s.a. The covalent binding of Haemophilus influenzae (Hib) PRP polysaccharide to TT may be carried out by the coupling chemistry developed by Chu et al (Infection and Immunity 1983, 40 (1); 245-256). Hib PRP polysaccharide is activated by adding CNBr and incubating at pH 10.5 for 6 minutes. The pH is lowered to pH 8.75 and adipic acid dihydrazide (ADH) is added and incubation continued for a further 90 minutes. The activated PRP may be coupled to, for instance, purified tetanus toxoid via carbodiimide condensation using 1-ethyl-3-(3-dimethyl-aminopropyl)carbodiimide (EDAC). EDAC is added to the activated PRP to reach a final ratio of 0.6 mg EDAC/mg activated PRP. The pH is adjusted to 5.0 and purified tetanus toxoid added to reach 2 mg TT/mg activated PRP. The resulting solution is left for three days with mild stirring. After filtration through a 0.45 μm membrane, the conjugate may be purified on a sephacryl S500HR (Pharmacia, Sweden) column equilibrated in 0.2M NaCl.

The Hib antigen conjugate may optionally be adsorbed onto aluminium phosphate as described in WO97/00697, or may be unadsorbed as described in WO02/00249 or may not have undergone a specific process for adsorption. By an antigen being ‘unadsorbed onto an aluminium adjuvant salt’ herein it is meant that an express or dedicated adsorption step for the antigen on fresh aluminium adjuvant salt is not involved in the process of formulating the composition. In one embodiment, Hib is present at a low dose (e.g. 1-6 μg, 2-4 μg or around or exactly 2.5 μg of saccharide) as described in WO 02/00249.

In one embodiment, the Hib saccharide conjugate is present in a lower saccharide dose than the saccharide dose of the Vi saccharide conjugate. For instance, the Hib saccharide conjugate may comprise 0.1-9, 1-5, or 2-3 μg of saccharide per human dose (normally 0.5 mL). The Hib saccharide may be conjugated to any protein carrier described herein, for example one selected from the group consisting of TT, DT, CRM197, fragment C of TT, protein D, OMPC and pneumolysin. In one aspect, the same protein carrier is used (e.g. independently) in the Hib saccharide conjugate and the Vi saccharide conjugate, for instance TT. The ratio of Hib saccharide to protein carrier in the Hib saccharide conjugate may be between 1:5 and 5:1 (w/w), for instance between 1:1 and 1:4, 1:2 and 1:3.5 or around 1:3 (w/w). The Hib saccharide may be conjugated to the protein carrier via a linker, which is typically bifunctional (homo or hetero bifunctional). The linker may have two reactive amino groups (one on each end), or two reactive carboxylic acid groups, or a reactive amino group at one end and a reactive carboxylic acid group at the other end. The linker may have between 4 and 12 carbon atoms. In one aspect the linker is ADH. The Hib saccharide may be conjugated to the protein carrier or linker using CNBr or CDAP. The protein carrier may be conjugated to the Hib saccharide or linker using carbodiimide chemistry, optionally EDAC chemistry. Further vaccine combinations involving the Vi and/or Hib conjugate antigen in which the Vi conjugates of the present invention may be used are described in PCT/EP2006/006210, PCT/EP2006/006188, PCT/EP2006/006269, PCT/EP2006/006268, or PCT/EP2006/006220.

In further aspects the Vi or Vi+Hib conjugates in the immunogenic compositions or vaccines of the invention are mixed with further antigens. For instance one or more from the following list may be added singly or in any combination (described in further detail below): a DTP (DTPa or DTPw) vaccine, a Hepatitis B vaccine/antigen such as hepatitis B surface antigen, optionally adsorbed onto aluminium phosphate, a Hepatitis A vaccine/antigen such as an inactivated hepatitis A virus preparation, a Polio virus vaccine/antigen such as an inactivated polio virus (IPV) preparation (optionally comprising types 1, 2 and 3), one or more meningococcal capsular saccharide—protein carrier conjugates [where the capsular saccharide(s) are derived from the following meningococcal serogroups: A, C, W135, Y, A and C, A and W135, A and Y, C and W135, C and Y, W135 and Y, A and C and W135, A and C and Y, A and W135 and Y, C and W135 and Y, A and C and W135 and Y], a malaria vaccine/antigen such as RTS,S.

In a further aspect the Vi and Hib capsular saccharide conjugates are co-lyophilised, optionally in the presence of a stabilising agent for example a polyol such as sucrose and/or trehalose. The lyophilised formulation may further comprise one or more meningococcal capsular saccharide—protein carrier conjugates (where the capsular saccharide(s) are derived from the following meningococcal serogroups: A, C, W135, Y, A and C, A and W135, A and Y, C and W135, C and Y, W135 and Y, A and C and W135, A and C and Y, A and W135 and Y, C and W135 and Y, A and C and W135 and Y). The lyophilised composition of the invention may be reconstituted with an aqueous medium prior to administration. The aqueous medium may be buffered. It may have further antigens for instance those listed above not already included in the lyophilised composition [e.g. one or more from the following list may be present singly or in any combination (described in further detail below) in the aqueous medium: a DTP (DTPa or DTPw) vaccine, a Hepatitis B vaccine/antigen such as hepatitis B surface antigen, optionally adsorbed onto aluminium phosphate, a Hepatitis A vaccine/antigen such as an inactivated hepatitis A virus preparation, a Polio virus vaccine/antigen such as an inactivated polio virus (IPV) preparation (optionally comprising types 1, 2 and 3), one or more meningococcal capsular saccharide—protein carrier conjugates [where the capsular saccharide(s) are derived from the following meningococcal serogroups: A, C, W135, Y, A and C, A and W135, A and Y, C and W135, C and Y, W135 and Y, A and C and W135, A and C and Y, A and W135 and Y, C and W135 and Y, A and C and W135 and Y], a malaria vaccine/antigen such as RTS,S].

The immunogenic composition or vaccines of the invention may contain aluminium phosphate, aluminium hydroxide or a mixture of both. Alternatively it may contain no aluminium salts, or may be unadjuvanted. The immunogenic composition or vaccine of the invention may be buffered at between pH 7.0 and 8.0.

In a further aspect of the invention a vaccine kit is provided for concomitant or sequential administration comprising two multi-valent immunogenic compositions for conferring protection in a host against disease caused by Bordetella pertussis, Clostridium tetani, Corynebacterium diphtheriae, Salmonella typhi and Haemophilus influenzae, said kit comprising a first container comprising:
tetanus toxoid (TT),
diphtheria toxoid (DT), and
whole cell or acellular pertussis components (Pw or Pa);
   and a second container comprising an immunogenic composition or vaccine of the invention. The first container may further comprise hepatitis B surface antigen, optionally adsorbed on aluminium phosphate. The first or second container may further comprise inactivated polio virus (IPV).

A use of the immunogenic composition or vaccine or kit of the invention in the manufacture of a medicament for the prevention or treatment of disease is also provided, as is method of preventing or treating disease comprising the step of administering an effective dose of the immunogenic composition or vaccine of the invention to a patient in need thereof. The use or method of the invention may be in respect of diseases caused by one or more bacteria selected from a list consisting of: N. meningitidis, Salmonella typhi, H. influenzae, Bordetella pertussis, Clostridium tetani, and Corynebacterium diphtheriae.

### Further Antigens/Vaccines for Addition to the Compositions/Vaccines of the Invention

### DTP Vaccine/Antigen Components

DTP vaccines are well known vaccines to prevent or treat diphtheria, tetanus and B. pertussis disease. The vaccines of the invention may comprise diphtheria, tetanus and/or pertussis component(s).

The diphtheria antigen is typically a diphtheria toxoid. The preparation of diphtheria toxoids (DT) is well documented. Any suitable diphtheria toxoid may be used. For instance, DT may be produced by purification of the toxin from a culture of Corynebacterium diphtheriae followed by chemical detoxification, but is alternatively made by purification of a recombinant, or genetically detoxified analogue of the toxin (for example, CRM197, or other mutants as described in U.S. Pat. No. 4,709,017, U.S. Pat. No. 5,843,711, U.S. Pat. No. 5,601,827, and U.S. Pat. No. 5,917,017). In one embodiment, the diphtheria toxoid of the invention may be adsorbed onto an aluminium salt such as aluminium hydroxide. In another embodiment, the diphtheria toxoid of the invention may be adsorbed onto an aluminium salt such as aluminium phosphate. In a further embodiment the diphtheria toxoid may be adsorbed onto a mixture of both aluminium hydroxide and aluminium phosphate.

The tetanus antigen of the invention is typically a tetanus toxoid. Methods of preparing tetanus toxoids (TT) are well known in the art. In one embodiment TT is produced by purification of the toxin from a culture of Clostridium tetani followed by chemical detoxification, but is alternatively made by purification of a recombinant, or genetically detoxified analogue of the toxin (for example, as described in EP 209281). Any suitable tetanus toxoid may be used. ‘Tetanus toxoid’ may encompass immunogenic fragments of the full-length protein (for instance Fragment C—see EP 478602). In one embodiment, the tetanus toxoid of the invention may be adsorbed onto an aluminium salt such as aluminium hydroxide. In another embodiment, the tetanus toxoid of the invention may be adsorbed onto an aluminium salt such as aluminium phosphate. In a further embodiment the tetanus toxoid may be adsorbed onto a mixture of both aluminium hydroxide and aluminium phosphate.

The pertussis component of the invention may be either acellular (Pa) where purified pertussis antigens are used or whole-cell (Pw) where killed whole cell pertussis is used as the pertussis component. Pw may be inactivated by several methods, including mercury free methods. Such methods may include heat (e.g. 55-65° C. or 56-60° C., for 5-60 minutes or 10-30 minutes, e.g. 60° C. for 30 minutes), formaldehyde (e.g. 0.1% at 37°, 24 hours), glutaraldehyde (e.g. 0.05% at room temperature, 10 minutes), acetone-I (e.g. three treatments at room temperature) and acetone-II (e.g. three treatments at room temperature and fourth treatment at 37° C.) inactivation (see for example Gupta et al., 1987, J. Biol. Stand. 15:87; Gupta et al., 1986, Vaccine, 4:185). Methods of preparing killed, whole-cell Bordetella pertussis (Pw) suitable for this invention are disclosed in WO 93/24148, as are suitable formulation methods for producing DT-TT-Pw-HepB vaccines. Thiomersal has been used in the past in the preparation of killed whole-cell Bordetella pertussis. However, in one embodiment it is not used in the formulation process of the vaccines of the present invention.

A Pw dose of 5-50 IOU, 7-40 IOU, 9-35 IOU, 11-30 IOU, 13-25 IOU, 15-21 IOU or around or exactly 20 IOU is typically used.

Acellular Pa vaccines are also well known, and may comprise 2 or more antigens from: pertussis toxoid [or known detoxified genetic mutants of pertussis toxin] (PT), filamentous haemagglutinin (FHA), pertactin (PRN), agglutinogens 2 & 3. In one embodiment, the Pa vaccine comprises PT, FHA and PRN.

In one embodiment, the pertussis component of the invention may be adsorbed onto an aluminium salt such as aluminium hydroxide. In another embodiment, the pertussis component of the invention may be adsorbed onto an aluminium salt such as aluminium phosphate. In a further embodiment the pertussis component may be adsorbed onto a mixture of both aluminium hydroxide and aluminium phosphate.

### Hepatitis B Antigen/Vaccine

The preparation of Hepatitis B surface antigen (HBsAg) is well documented. See for example, Hartford et al., 1983, Develop. Biol. Standard 54:125, Gregg et al., 1987, Biotechnology 5:479, EP0226846, EP0299108. It may be prepared as follows. One method involves purifying the antigen in particulate form from the plasma of chronic hepatitis B carriers, as large quantities of HBsAg are synthesised in the liver and released into the blood stream during an HBV infection. Another method involves expressing the protein by recombinant DNA methods. The HBsAg may be prepared by expression in the Saccharomyces cerevisiae yeast, pichia, insect cells (e.g. Hi5) or mammalian cells. The HBsAg may be inserted into a plasmid, and its expression from the plasmid may be controlled by a promoter such as the “GAPDH” promoter (from the glyceraldehyde-3-phosphate dehydrogenase gene). The yeast may be cultured in a synthetic medium. HBsAg can then be purified by a process involving steps such as precipitation, ion exchange chromatography, and ultrafiltration. After purification, HBsAg may be subjected to dialysis (e.g. with cysteine). The HBsAg may be used in a particulate form.

As used herein the expression “Hepatitis B surface antigen” or “HBsAg” includes any HBsAg antigen or fragment thereof displaying the antigenicity of HBV surface antigen. It will be understood that in addition to the 226 amino acid sequence of the HBsAg S antigen (see Tiollais et al., 1985, Nature 317:489 and references therein) HBsAg as herein described may, if desired, contain all or part of a pre-S sequence as described in the above references and in EP0278940. In particular, the HBsAg may comprise a polypeptide comprising an amino acid sequence comprising residues 133-145 followed by residues 175-400 of the L-protein of HBsAg relative to the open reading frame on a Hepatitis B virus of ad serotype (this polypeptide is referred to as L*; see EP0414374). HBsAg within the scope of the invention may also include the preS1-preS2-S polypeptide described in EP0198474 (Endotronics) or analogues thereof such as those described in EP0304578 (McCormick and Jones) HBsAg as herein described can also refer to mutants, for example the “escape mutant” described in WO 91/14703 or EP0511855A1, especially HBsAg wherein the amino acid substitution at position 145 is to arginine from glycine.

The HBsAg may be in particle form. The particles may comprise for example S protein alone or may be composite particles, for example L*, S) where L* is as defined above and S denotes the S-protein of HBsAg. The said particle is advantageously in the form in which it is expressed in yeast.

In one embodiment, HBsAg is the antigen used in EngerixB™ (GlaxoSmithKline Biologicals S.A.), which is further described in WO93/24148.

Hepatitis B surface antigen may be adsorbed onto aluminium phosphate, which may be done before mixing with the other components (described in WO93/24148). The Hepatitis B component should be substantially thiomersal free (method of preparation of HBsAg without thiomersal has been previously published in EP1307473).

Neisseria meningitidis Types A, C, W or Y Antigens

The vaccines/compositions of the invention may further comprise a capsular saccharide of a bacterium selected from the group consisting of N. meningitidis type A (MenA, optionally conjugated to a carrier protein), N. meningitidis type C (MenC, optionally conjugated to a carrier protein), N. meningitidis type W (MenW, optionally conjugated to a carrier protein), and N. meningitidis type Y (MenY, optionally conjugated to a carrier protein).

The vaccines of the invention may comprise one or more antigens from the different strains of N. meningitidis, which may be used alone or in any combination of two, three or four components as detailed below: MenA, MenC, MenW, MenY, or MenA+MenC, MenA+MenW, MenA+MenY, MenC+MenW, MenC+MenY, MenW+MenY or MenA+MenC+MenW, MenA+MenC+MenY, MenA+MenW+MenY, MenC+MenW+MenY or MenA+MenC+MenW+MenY.

In one embodiment, the Neisseria meningitidis component(s) of the invention may be adsorbed onto an aluminium salt such as aluminium hydroxide. In another embodiment, the Neisseria meningitidis component(s) of the invention may be adsorbed onto an aluminium salt such as aluminium phosphate. In a further embodiment the Neisseria meningitidis component(s) may be adsorbed onto a mixture of both aluminium hydroxide and aluminium phosphate. In one embodiment the Neisseria meningitidis component(s) may be unadsorbed onto an adjuvant, e.g. an aluminium adjuvant salt. The conjugates may be made by any means—in one embodiment the methods described in PCT/EP2006/006210, PCT/EP2006/006188, PCT/EP2006/006269, PCT/EP2006/006268, or PCT/EP2006/006220 are utilised. The conjugates may be made using the conjugation process of the present invention.

Neisseria meningitidis Type B Bleb or Antigen(s)

The vaccines of the invention may also comprise a MenB component such as an outer membrane vesicle or bleb as described in WO01/09350, WO03/105890, WO04/014417, or WO04/014418 or a conjugated MenB capsular saccharide (or derivative thereof) antigen (e.g. see WO 96/40239). In one embodiment, the MenB component(s) of the invention may be adsorbed onto an aluminium salt such as aluminium hydroxide. In another embodiment, the MenB component(s) of the invention may be adsorbed onto an aluminium salt such as aluminium phosphate. In a further embodiment the MenB component(s) may be adsorbed onto a mixture of both aluminium hydroxide and aluminium phosphate. In one embodiment the MenB component(s) may be unadsorbed onto an adjuvant, e.g. an aluminium adjuvant salt.

### Hepatitis A Antigen(s)/Vaccines

A component affording protection against Hepatitis A may be the product known as Havrix™ (Registered Trade Mark of GlaxoSmithKline Biologicals S.A.) which is a killed attenuated vaccine derived from the HM-175 strain of Hepatitis A virus (HAV) (see “Inactivated Candidate Vaccines for Hepatitis A” by F. E. Andre et al., 1980, Prog. Med. Virol. 37:72 and the product monograph “Havrix” published by SmithKline Beecham Biologicals 1991). Flehmig et al. (1990, Prog. Med Virol. 37:56) have reviewed the clinical aspects, virology, immunology and epidemiology of Hepatitis A and discussed approaches to the developments of vaccines against this common viral infection. As used herein the expression “HAV antigen” or “HAV vaccine” or “Hepatitis A vaccine” refers to any antigen capable of stimulating neutralising antibody to HAV in humans. In one embodiment the HAV antigen comprises inactivated attenuated virus particles, or in another embodiment it may be a HAV capsid or HAV viral protein, which may conveniently be obtained by recombinant DNA technology. In one embodiment, the Hepatitis A component of the invention may be adsorbed onto an aluminium salt such as aluminium hydroxide. In another embodiment, the Hepatitis A component of the invention may be adsorbed onto an aluminium salt such as aluminium phosphate. In a further embodiment the Hepatitis A component may be adsorbed onto a mixture of both aluminium hydroxide and aluminium phosphate. In one embodiment the compositions of the invention comprising a Hepatitis A vaccine do not comprise phenol.

### Malarial Antigen(s)/Vaccines

The vaccines of the invention may further comprise Malarial antigen(s). The Malarial antigen may be RTS,S (hybrid protein between CS and HBsAg—described in U.S. Pat. No. 6,306,625 and EP 0614465). In one embodiment, RTS,S may be used in the vaccines of the invention in place of HBsAg. Other Malarial antigens may also be used in the vaccines of the invention, including CS protein, RTS, TRAP, 16 kD protein of B 2992, AMA-1, MSP1, optionally including CpG (WO2006/029887, WO98/05355, WO01/00231).

In one embodiment, the Malarial antigen(s) of the invention may be adsorbed onto an aluminium salt such as aluminium hydroxide. In another embodiment, the Malarial antigen(s) of the invention may be adsorbed onto an aluminium salt such as aluminium phosphate. In a further embodiment the Malarial antigen(s) may be adsorbed onto a mixture of both aluminium hydroxide and aluminium phosphate. In one embodiment the Malarial antigen is adjuvanted with an oil-in-water emulsion and/or lipid A derivative (such as MPL) and or sterol (such as cholesterol) and/or tocol (such as α-tocopherol) In another embodiment the Malaria antigen(s) may be unadsorbed onto an adjuvant, e.g. an aluminium adjuvant salt.

### Polio Virus Antigen(s)/Vaccines

The vaccines of the invention may further comprise antigens affording protection against polio virus. In one embodiment Inactivated Polio Virus (IPV) is included. Vaccines/compositions of the invention may include IPV type 1 (e.g. Mahoney or Brunhilde) or IPV type 2 (e.g. MEF-1) or IPV type 3 (e.g. Saukett), or IPV types 1 and 2, or IPV types 1 and 3, or IPV types 2 and 3, or IPV types 1, 2 and 3.

Methods of preparing inactivated poliovirus (IPV) are well known in the art. In one embodiment, IPV should comprise types 1, 2 and 3 as is common in the vaccine art, and may be the Salk polio vaccine which is inactivated with formaldehyde (see for example, Sutter et al., 2000, Pediatr. Clin. North Am. 47:287; Zimmerman & Spann 1999, Am Fam Physician 59:113; Salk et al., 1954, Official Monthly Publication of the American Public Health Association 44(5):563; Hennesen, 1981, Develop. Biol. Standard 47:139; Budowsky, 1991, Adv. Virus Res. 39:255).

In one embodiment the IPV is not adsorbed (e.g. before mixing with other components). In another embodiment, the IPV component(s) of the invention may be adsorbed onto an aluminium salt such as aluminium hydroxide (e.g. before or after mixing with other components). In another embodiment, the IPV component(s) of the invention may be adsorbed onto an aluminium salt such as aluminium phosphate. In a further embodiment the IPV component(s) may be adsorbed onto a mixture of both aluminium hydroxide and aluminium phosphate. If adsorbed, one or more IPV components may be adsorbed separately or together as a mixture. IPV may be stabilised by a particular drying process as described in WO2004/039417.

Poliovirus may be grown in cell culture. The cell culture may be a VERO cell line or PMKC, which is a continuous cell line derived from monkey kidney. VERO cells can conveniently be cultured microcarriers. Culture of the VERO cells before and during viral infection may involve the use of bovine-derived material, such as calf serum, and this material should be obtained from sources which are free from bovine spongiform encephalitis (BSE). Culture may also involve materials such as lactalbumin hydrolysate. After growth, virions may be purified using techniques such as ultrafiltration, diafiltration, and chromatography. Prior to administration to patients, the viruses must be inactivated, and this can be achieved by treatment with formaldehyde.

Viruses may be grown, purified and inactivated individually, and then combined to give a bulk mixture for IPV vaccine use or for addition to the other antigens.

Antigens in vaccines of the invention will be present in “immunologically effective amounts” i.e. the administration of that amount to an individual, either in a single dose or as part of a series, is effective for treatment or prevention of disease. Dosage treatment may be a single dose schedule or a multiple dose schedule (e.g. including booster doses).

Standard doses of available polio vaccines contain 40 D antigen units of inactivated poliovirus type 1, 8 D antigen units of inactivated poliovirus type 2 and 32 D antigen units of inactivated poliovirus type 3 (e.g. Infanrix-IPV™).

### Adjuvants

The vaccines/compositions of the invention may include a pharmaceutically acceptable excipient such as a suitable adjuvant. Suitable adjuvants include an aluminium salt such as aluminium hydroxide or aluminium phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL [3D-MPL], quil A, Saponin, QS21, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, bioadhesives and mucoadhesives, microparticles, liposomes, polyoxyethylene ether formulations, polyoxyethylene ester formulations, muramyl peptides or imidazoquinolone compounds (e.g. imiquamod and its homologues). Human immunomodulators suitable for use as adjuvants in the invention include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte, macrophage colony stimulating factor (GM-CSF) may also be used as adjuvants.

In one embodiment of the invention, the adjuvant composition of the formulations induces an immune response predominantly of the TH1 type. High levels of TH1-type cytokines (e.g. IFN-γ, TNFα, IL-2 and IL-12) tend to favour the induction of cell mediated immune responses to an administered antigen. Within one embodiment, in which a response is predominantly TH1-type, the level of TH1-type cytokines will increase to a greater extent than the level of TH2-type cytokines. The levels of these cytokines may be readily assessed using standard assays. For a review of the families of cytokines, see Mosmann and Coffman, 1989, Ann. Rev. Immunol. 7:145.

Accordingly, suitable adjuvant systems which promote a predominantly TH1 response include, derivatives of lipid A (e.g. of reduced toxicity), Monophosphoryl lipid A (MPL) or a derivative thereof, particularly 3-de-O-acylated monophosphoryl lipid A (3D-MPL), and a combination of monophosphoryl lipid A, optionally 3-de-O-acylated monophosphoryl lipid A together with an aluminium salt. An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210. The vaccine may additionally comprise a saponin, which may be QS21. The formulation may also comprise an oil in water emulsion and tocopherol (WO 95/17210). Unmethylated CpG containing oligonucleotides (WO 96/02555) are also preferential inducers of a TH1 response and are suitable for use in the present invention.

The vaccines of the invention may also comprise combinations of aspects of one or more of the adjuvants identified above.

Al(OH)₃/AlPO₄ ratios may be 0/115, 23/92, 69/46, 46/69, 92/23 or 115/0.

Alternatively certain components of the vaccines of the invention may be not expressly adsorbed onto adjuvant, in particular aluminium salts.

IPV may be unadsorbed or adsorbed onto Al(OH)₃, DT may be adsorbed onto Al(OH)₃ or AlPO₄, TT may be adsorbed onto Al(OH)₃ or AlPO₄, Pw may be adsorbed onto or mixed with AlPO₄, PRN may be adsorbed onto Al(OH)₃, FHA may be adsorbed onto Al(OH)₃, PT may be adsorbed onto Al(OH)₃, HB (HepB surface antigen) may be adsorbed onto AlPO₄, Hib may be adsorbed onto AlPO₄ or unadsorbed, Men ACWY may be adsorbed onto Al(OH)₃ or AlPO₄ or unadsorbed, MenB may be adsorbed onto Al(OH)₃ or AlPO₄ or unadsorbed, Vi may be adsorbed onto Al(OH)₃ or AlPO₄ or unadsorbed, HepA may be adsorbed onto Al(OH)₃ or AlPO₄.

Antigens which are preadsorbed onto an aluminium salt can be preadsorbed individually prior to mixing. In another embodiment, a mix of antigens may be preadsorbed prior to mixing with further adjuvants. In one embodiment, IPV may be adsorbed separately or as a mixture of IPV types 1, 2 and 3.

The meaning of “adsorbed antigen” is taken to mean greater than 30%, 40%, 50%, 60%, 70%, 80%, or 90% adsorbed.

The meaning of the terms “aluminium phosphate” and “aluminium hydroxide” as used herein includes all forms of aluminium hydroxide or aluminium phosphate which are suitable for adjuvanting vaccines. For example, aluminium phosphate can be a precipitate of insoluble aluminium phosphate (amorphous, semi-crystalline or crystalline), which can be optionally but not exclusively prepared by mixing soluble aluminium salts and phosphoric acid salts. “Aluminium hydroxide” can be a precipitate of insoluble (amorphous, semi-crystalline or crystalline) aluminium hydroxide, which can be optionally but not exclusively prepared by neutralising a solution of aluminium salts. Particularly suitable are the various forms of aluminium hydroxide and aluminium phosphate gels available from commercial sources for example, Alhydrogel (aluminium hydroxide, 3% suspension in water) and Adjuphos (aluminium phosphate, 2% suspension in saline) supplied by Brenntag Biosector (Denmark).

### Non-Immunological Components of Vaccines of the Invention

Vaccines of the invention will typically, in addition to the antigenic and adjuvant components mentioned above, comprise one or more “pharmaceutically acceptable carriers or excipients”, which include any excipient that does not itself induce the production of antibodies harmful to the individual receiving the composition. Suitable excipients are typically large, slowly metabolised macromolecules such as proteins, saccharides, polylactic acids, polyglycolic acids, polymeric amino acids, amino acid copolymers, sucrose (Paoletti et al., 2001, Vaccine, 19:2118), trehalose (WO 00/56365), lactose and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier. A thorough discussion of pharmaceutically acceptable excipients is available in reference Gennaro, 2000, Remington: The Science and Practice of Pharmacy, 20^{th} edition, ISBN:0683306472.

Compositions of the invention may be lyophilised or in aqueous form, i.e. solutions or suspensions. Liquid formulations of this type allow the compositions to be administered direct from their packaged form, without the need for reconstitution in an aqueous medium, and are thus ideal for injection. Compositions may be presented in vials, or they may be presented in ready filled syringes. The syringes may be supplied with or without needles. A syringe will include a single dose of the composition, whereas a vial may include a single dose or multiple doses (e.g. 2 doses).

Liquid vaccines of the invention are also suitable for reconstituting other vaccines from a lyophilised form. Where a vaccine is to be used for such extemporaneous reconstitution, the invention provides a kit, which may comprise two vials, or may comprise one ready-filled syringe and one vial, with the contents of the syringe being used to reconstitute the contents of the vial prior to injection.

Vaccines of the invention may be packaged in unit dose form or in multiple dose form (e.g. 2 doses). For multiple dose forms, vials are preferred to pre-filled syringes. Effective dosage volumes can be routinely established, but a typical human dose of the composition for injection has a volume of 0.5 mL.

In one embodiment, vaccines of the invention have a pH of between 6.0 and 8.0, in another embodiment vaccines of the invention have a pH of between 6.3 and 6.9, e.g. 6.6±0.2. Vaccines may be buffered at this pH. Stable pH may be maintained by the use of a buffer. If a composition comprises an aluminium hydroxide salt, a histidine buffer may be used (WO03/009869). The composition should be sterile and/or pyrogen free.

Compositions of the invention may be isotonic with respect to humans.

Vaccines of the invention may include an antimicrobial, particularly when packaged in a multiple dose format. Thiomersal should be avoided as this leads to loss of potency of the IPV component. Other antimicrobials may be used, such as 2-phenoxyethanol or parabens (methyl, ethyl, propyl parabens). Any preservative is preferably present at low levels. Preservative may be added exogenously and/or may be a component of the bulk antigens which are mixed to form the composition (e.g. present as a preservative in pertussis antigens).

In one embodiment, vaccines of the invention are thiomersal free or substantially thiomersal free. By thiomersal free or substantially thiomersal free it is meant that there is not enough thiomersal present in the final formulation to negatively impact the potency of the IPV component. For instance, if thiomersal is used during the Pw or Hepatitis B surface antigen purification process it should be substantially removed prior to mixture with IPV. Thiomersal content in the final vaccine should be less than 0.025 μg/μg protein, 0.02 μg/μg protein, 0.01 μg/μg protein or 0.001 μg/μg protein, for instance 0 μg/μg protein. In one embodiment, thiomersal is not added nor used in the purification of any component. See for instance EP1307473 for Hepatitis B and see above for Pw processes where killing is achieved not in the presence of thiomersal.

Vaccines of the invention may comprise detergent e.g. a Tween (polysorbate), such as Tween 80. Detergents are generally present at low levels e.g. <0.01%.

Vaccines of the invention may include sodium salts (e.g. sodium chloride) to give tonicity. The composition may comprise sodium chloride. In one embodiment, the concentration of sodium chloride in the composition of the invention is in the range of 0.1 to 100 mg/mL (e.g. 1-50 mg/mL, 2-20 mg/mL, 5-15 mg/mL) and in a further embodiment the concentration of sodium chloride is 10±2 mg/mL NaCl e.g. about 9 mg/mL.

Vaccines of the invention will generally include a buffer. A phosphate or histidine buffer is typical.

Vaccines of the invention may include free phosphate ions in solution (e.g. by the use of a phosphate buffer) in order to favour non-adsorption of antigens. The concentration of free phosphate ions in the composition of the invention is in one embodiment between 0.1 and 10.0 mM, or in another embodiment between 1 and 5 mM, or in a further embodiment about 2.5 mM.

### Vaccine Formulations

In one embodiment, the vaccines of the invention are formulated as a vaccine for in vivo administration to the host, such that they confer an antibody titre superior to the criterion for seroprotection for each antigenic component for an acceptable percentage of human subjects. This is an important test in the assessment of a vaccine's efficacy throughout the population. Antigens with an associated antibody titre above which a host is considered to be seroconverted against the antigen are well known, and such titres are published by organisations such as WHO. In one embodiment, more than 80% of a statistically significant sample of subjects is seroconverted, in another embodiment more than 90% of a statistically significant sample of subjects is seroconverted, in a further embodiment more than 93% of a statistically significant sample of subjects is seroconverted and in yet another embodiment 96-100% of a statistically significant sample of subjects is seroconverted.

The amount of antigen in each vaccine dose is selected as an amount which induces an immunoprotective response without significant, adverse side effects in typical vaccines. Such amount will vary depending on which specific immunogens are employed. Generally it is expected that each dose will comprise 1-1000 μg of total immunogen, or 1-100 μg, or 1-40 μg, or 1-5 μg. An optimal amount for a particular vaccine can be ascertained by studies involving observation of antibody titres and other responses in subjects. A primary vaccination course may include 2-3 doses of vaccine, given one to two months apart, e.g. following the WHO recommendations for DTP immunisation.

### Packaging of Vaccines of the Invention

Vaccines of the invention can be packaged in various types of container e.g. in vials, in syringes, etc. A multidose vial will typically comprise a re-sealable plastic port through which a sterile needle can be inserted to remove a dose of vaccine, which reseals once the needle has been removed.

The vaccine may be supplied in various containers (e.g. 2 or 3). The contents of the containers may be mixed extemporaneously before administering to a host in a single injection or it may be administered concomitantly at different sites. The dose of the vaccine will typically be 0.5 mL.

In one embodiment of this aspect of the invention there is provided a kit comprising two multi-valent vaccines for conferring protection in a host against disease caused by poliovirus, Bordetella pertussis, Clostridium tetani, Corynebacterium diphtheriae and optionally one or more of Hepatitis B, Haemophilus influenza type B, Neisseria meningitidis type A, Neisseria meningitidis type C, Neisseria meningitidis type W, Neisseria meningitidis type Y, Neisseria meningitidis type B, Salmonella typhi, Hepatitis A or Malaria.

The kit comprises a first container comprising:
(1) (a) optionally Inactivated polio virus (IPV),
   (b) diphtheria toxoid (DT or D),
   (c) tetanus toxoid (TT or T),
   (d) killed whole-cell Bordetella pertussis (Pw) or 2 or more acellular pertussis components (Pa) (see above),
   (e) optionally Hepatitis B surface antigen (HepB or HB),
   (f) optionally a conjugate of a carrier protein and the capsular saccharide of H. influenzae type B (Hib),
   (g) optionally either or both conjugates of a carrier protein and a capsular saccharide of a N. meningitidis type A (MenA) or N. meningitidis type C (MenC) [e.g. made by the conjugation process of the invention], and
      a second container comprising:
(2A) (a) conjugates of a carrier protein and a capsular saccharide N. meningitidis type A (MenA), N. meningitidis type C (MenC), N. meningitidis type W (MenW) and/or N. meningitidis type Y (MenY) (see above for various Men saccharide combinations of the invention) [e.g. made by the conjugation process of the invention], and
   (b) optionally a conjugate of a carrier protein and the capsular saccharide of H. influenzae type B (Hib); or
(2B) (a) a conjugate of a carrier protein and the capsular saccharide of H. influenzae type B (Hib), and
   (b) a conjugate of a carrier protein and Vi saccharide of Salmonella typhi made by the conjugation process of the invention

The containers may in either case additionally comprise HepA antigen(s) and/or MenB antigen(s) and/or RTS,S and/or Streptococcus pneumonia antigen(s).

In either case, the same antigen should not be present in both containers.

In one embodiment the first container has in addition to components b), c), d) also a), e), f), g), e)+f), e)+g), f)+g) or e)+f)+g), a)+e), a)+f), a)+g), a)+e)+f), a)+e)+g), a)+f)+g), a)+e)+f)+g).

In one embodiment the vaccine of the first container may be liquid and the vaccine of the second container may be either liquid or lyophilised (e.g. in the presence of a known stabilising excipient such as sucrose or trehalose).

The containers of the kit can be packaged separately or, optionally, packed together. In one embodiment, the kit is provided with a list of instructions for administration of the vaccines in the two or more containers.

In one embodiment, where a container in a kit contains a certain saccharide conjugate, the same conjugate is not present in the other containers of the kit.

In one embodiment the vaccines of the first and second containers are administered concomitantly at different sites (as described below under “administration of vaccines of the invention), and in an alternative embodiment the inventors envision that the contents of the first and second containers may be mixed (optionally extemporaneously) before administration as a single vaccine.

### Preparing Vaccines of the Invention

The present invention also provides a method for producing a vaccine formulation comprising the step of mixing the components of the vaccine together with a pharmaceutically acceptable excipient.

In one embodiment of the present invention there is provided a vaccine as herein described for use in a medicament for the treatment or prevention of diseases caused by infection by one or more of poliovirus, Bordetella pertussis, Clostridium tetani, Corynebacterium diphtheriae, Hepatitis B virus, Haemophilus influenzae, Neisseria meningitidis type A, Neisseria meningitidis type C, Neisseria meningitidis type W, Neisseria meningitidis type Y, Salmonella typhi or Hepatitis A.

In another embodiment of the invention there is provided a use of the vaccines of the invention in the manufacture of a medicament for the treatment or prevention of diseases caused by infection by one or more of poliovirus, Bordetella pertussis, Clostridium tetani, Corynebacterium diphtheriae, Hepatitis B virus, Haemophilus influenzae, Neisseria meningitidis type A, Neisseria meningitidis type C, Neisseria meningitidis type W, Neisseria meningitidis type Y, Salmonella typhi or Hepatitis A.

Additionally, a method of immunising a human host against disease caused by one or more of poliovirus, Bordetella pertussis, Clostridium tetani, Corynebacterium diphtheriae, Hepatitis B virus, Haemophilus influenzae, Neisseria meningitidis type A, Neisseria meningitidis type C, Neisseria meningitidis type W, Neisseria meningitidis type Y, Salmonella typhi or Hepatitis A, which method comprises administering to the host an immunoprotective dose of the vaccine of the invention is also provided.

The amount of antigen in each vaccine dose is selected as an amount which induces an immunoprotective response without significant, adverse side effects in typical vaccines. Such amount will vary depending upon which specific immunogen is employed and how it is presented. In one embodiment each dose will comprise 0.1-100 μg of saccharide, in another embodiment each dose will comprise 0.1-50 μg, in a further embodiment each dose will comprise 0.1-10 μg, in yet another embodiment each dose will comprise 1 to 5 μg saccharide.

In one embodiment, the content of protein antigens in the vaccine will be in the range 1-100 μg, in another embodiment the content of the protein antigens in the vaccines will be in the range 5-50 μg, in a further embodiment the content of the protein antigens in the vaccines will be in the range 5-25 μg.

Vaccine preparation is generally described in Vaccine Design [“The subunit and adjuvant approach” (eds Powell M. F. & Newman M. J.) (1995) Plenum Press New York]. Encapsulation within liposomes is described by Fullerton, U.S. Pat. No. 4,235,877. Conjugation of proteins to macromolecules is disclosed, for example by Likhite, U.S. Pat. No. 4,372,945 and by Armor et al., U.S. Pat. No. 4,474,757. Use of Quil A is disclosed by Dalsgaard et al., 1977, Acta Vet Scand. 18:349. 3D-MPL is available from Ribi immunochem, USA and is disclosed in British Patent Application No. 2220211 and U.S. Pat. No. 4,912,094. QS21 is disclosed in U.S. Pat. No. 5,057,540.

In one embodiment the amount of saccharide conjugates per 0.5 mL dose of bulk vaccine is less than 10 μg (of saccharide in the conjugate), in another embodiment the amount of conjugate is 1-7, in another embodiment the amount of conjugate is 2-6 μg, or in a further embodiment about 2.5, 3, 4 or 5 μg.

It will be appreciated that certain components, for example DTPw components, can be combined separately before adding the adsorbed HBsAg or other components.

In general, the combined vaccine compositions according to any aspect of the invention can be prepared as follows: The IPV, DTPw, HepB, MenA, MenC, MenW, MenY, MenB, Vi, Hepatitis A or other components are pre-adsorbed onto a suitable adjuvant, especially aluminium hydroxide or aluminium phosphate or a mixture of both. After allowing time for complete and stable adsorption of the respective components, the different components are combined under appropriate conditions. The Hib, Vi, MenA, MenC, MenW and/or MenY conjugate(s) may or may not be adsorbed onto aluminium adjuvant salt before being mixed with the DTPw vaccine.

In one embodiment, vaccines of the invention are prepared at between 15° C. and 30° C. (e.g. between 19° C. and 27° C., or at 23±4° C.).

### Administration of Vaccines of the Invention

The invention provides a method for raising an immune response in a mammal, comprising the step of administering an effective amount of a vaccine of the invention. The vaccines can be administered prophylactically (i.e. to prevent infection) or therapeutically (i.e. to treat disease after infection). The immune response is preferably protective and preferably involves antibodies. The method may raise a booster response.

Following an initial vaccination, subjects may receive one or several booster immunisations adequately spaced. Dosing treatment can be a single dose schedule or a multiple dose schedule. Multiple doses may be used in a primary immunisation schedule and/or in a booster immunisation schedule. A primary dose schedule, which may be in the first year of life, may be followed by a booster dose schedule. Suitable timing between priming doses (e.g. between 4-16 weeks), and between priming and boosting can be routinely determined.

In one embodiment, the mammal is a human. Where the vaccine is for prophylactic use, the human is preferably a child (e.g. a toddler of infant) or a teenager; where the vaccine is for therapeutic use, the human is preferably an adult. A vaccine intended for children may also be administered to adults e.g. to assess safety, dosage, immunogenicity, etc.

The vaccine preparations of the present invention may be used to protect or treat a mammal susceptible to infection, by means of administering said vaccine directly to a patient. Direct delivery may be accomplished by parenteral injection (intramuscularly, intraperitoneally, intradermally, subcutaneously, intravenously, or to the interstitial space of a tissue); or by rectal, oral, vaginal, topical, transdermal, intranasal, ocular, aural, pulmonary or other mucosal administration. In one embodiment, administration is by intramuscular injection to the thigh or the upper arm. Injection may be via a needle (e.g. a hypodermic needle), but needle free injection may alternatively be used. A typical intramuscular dose is 0.5 mL.

Bacterial infections affect various areas of the body and so the compositions of the invention may be prepared in various forms. For example, the compositions may be prepared as injectables, either as liquid solutions or suspensions. The composition may be prepared for pulmonary administration e.g. as an inhaler, using a fine powder or spray. The composition may be prepared as a suppository or pessary. The composition may be prepared for nasal, aural or ocular administration e.g. as spray, drops, gel or powder (see e.g. Almeida & Alpar, 1996, J Drug Targeting, 3:455; Bergquist et al., 1998, APMIS, 106:800). Successful intranasal administration of DTP vaccines has been reported (Ryan et al., 1999, Infect. Immun., 67:6270; Nagai et al., 2001, Vaccine, 19:4824).

In one embodiment the vaccines of the first and second (and third where applicable) containers are administered concomitantly at different sites, and in an alternative embodiment the inventors envision that the contents of the first and second containers may be mixed (optionally extemporaneously) before administration as a single vaccine.

The invention may be used to elicit systemic and/or mucosal immunity.

One way of checking the efficacy of therapeutic treatment involves monitoring bacterial infection after administration of the composition of the invention. One way of checking efficacy of prophylactic treatment involves monitoring immune responses against the antigens after administration of the composition. Immunogenicity of compositions of the invention can be determined by administering them to test subjects (e.g. children 12-16 months age, or animal models—WO 01/30390) and then determining standard immunological parameters. These immune responses will generally be determined around 4 weeks after administration of the composition, and compared to values determined before administration of the composition. Rather than assessing actual protective efficacy in patients, standard animal and in vitro models and correlates of protection for assessing the efficacy of DTP vaccines are well known.

The terms “comprising”, “comprise” and “comprises” herein are intended by the inventors to be optionally substitutable with the terms “consisting of”, “consist of” and “consists of”, respectively, in every instance. The term “immunogenic composition” may be substituted for the term “vaccine” herein and vice versa.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLES

### Example 1

### Preparation of Polysaccharide Conjugates

### Example 1a

### Preparation of Meningococcal MenA and MenC Capsular Polysaccharide Conjugate According to the Invention

MenC-TT conjugates were produced using native polysaccharides (of over 150 kDa as measured by MALLS) or were slightly microfluidised. MenA-TT conjugates were produced using either native polysaccharide or slightly microfluidised polysaccharide of over 60 kDa as measured by the MALLS method of example 2. Sizing was by microfluidisation using a homogenizer Emulsiflex C-50 apparatus. The polysaccharides were then filtered through a 0.2 μm filter.

In order to conjugate MenA capsular polysaccharide to tetanus toxoid via a spacer, the following method was used. The covalent binding of the polysaccharide and the spacer (ADH) is carried out by a coupling chemistry by which the polysaccharide is activated under controlled conditions by a cyanylating agent, 1-cyano-4-dimethylamino-pyridinium tetrafluoroborate (CDAP). The spacer reacts with the cyanylated PS through its hydrazino groups, to form a stable isourea link between the spacer and the polysaccharide.

A 10 mg/ml solution of MenA (pH 6.0) [3.5 g] was treated with a freshly prepared 100 mg/ml solution of CDAP in acetonitrile/water (50/50 (v/v)) to obtain a CDAP/MenA ratio of 0.75 (w/w). After 1.5 minutes, the pH was raised to pH 10.0. Three minutes later, ADH was added to obtain an ADH/MenA ratio of 8.9. The pH of the solution was decreased to 8.75 and the reaction proceeded for 2 hours maintaining this pH (with temperature kept at 25° C.).

The PSA_{AH} solution was concentrated to a quarter of its initial volume and then diafiltered with 30 volumes of 0.2M NaCl using a Filtron Omega membrane with a cut-off of 10 kDa, and the retentate was filtered.

Prior to the conjugation (carbodiimide condensation) reaction, the purified TT solution and the PSA_{AH} solution were diluted to reach a concentration of 10 mg/ml for PSA_{AH} and 10 mg/ml for TT.

EDAC (1-ethyl-3-(3-dimethyl-aminopropyl) carbodiimide) was added to the PS_{AH} solution (2 g saccharide) in order to reach a final ratio of 0.9 mg EDAC/mg PSA_{AH}. The pH was adjusted to 5.0. The purified tetanus toxoid was added with a peristaltic pump (in 60 minutes) to reach 2 mg TT/mg PSA_{AH}. The resulting solution was left 60 min at +25° C. under stirring to obtain a final coupling time of 120 min. The solution was neutralised by addition of 1M Tris-Hcl pH 7.5 (1/10 of the final volume) and left 30 minutes at +25° C. then overnight at +2° C. to +8° C.

The conjugate was clarified using a 10 μm filter and was purified using a Sephacryl S400HR column (Pharmacia, Sweden). The column was equilibrated in 10 mM Tris-HCl (pH 7.0), 0.075 M NaCl and the conjugate (approx. 660 mL) was loaded on the column (+2° C. to +8° C.). The elution pool was selected as a function of optical density at 280 nm. Collection started when absorbance increased to 0.05. Harvest continued until the Kd reached 0.30. The conjugate was filter sterilised at +20° C., then stored at +2° C. to +8° C. The resultant conjugate had a polysaccharide:protein ratio of 1:2-1:4 (w/w).

In order to conjugate MenC capsular polysaccharide to tetanus toxoid via a spacer, the following method was used. The covalent binding of the polysaccharide and the spacer (ADH) is carried out by a coupling chemistry by which the polysaccharide is activated under controlled conditions by a cyanylating agent, 1-cyano-4-dimethylamino-pyridinium tetrafluoroborate (CDAP). The spacer reacts with the cyanylated PS through its hydrazino groups, to form a stable isourea link between the spacer and the polysaccharide.

A 20 mg/ml solution of MenC (pH6.0) (3.5 g) was treated with a freshly prepared 100 mg/ml solution of CDAP in acetonitrile/water (50/50 (v/v)) to obtain a CDAP/MenC ratio of 1.5 (w/w). After 1.5 minutes, the pH was raised to pH 10.0. At activation pH 5M NaCl was added to achieve a final concentration of 2M NaCl. Three minutes later, ADH was added to obtain an ADH/MenC ratio of 8.9. The pH of the solution was decreased to 8.75 and the reaction proceeded for 2 hours (retained at 25° C.).

The PSC_{AH} solution was concentrated to a minimum of 150 mL and then diafiltered with 30 volumes of 0.2M NaCl using a Filtron Omega membrane with a cut-off of 10 kDa, and the retentate was filtered.

Prior to the conjugation reaction, the purified TT solution and the PSC_{AH} solution (2 g scale) were diluted in 0.2M NaCl to reach a concentration of 15 mg/ml for PSC_{AH} and 20 mg/ml for TT.

The purified tetanus toxoid was added to the PSC_{AH} solution in order to reach 2 mg TT/mg PSC_{AH}. The pH was adjusted to 5.0. EDAC (16.7 mg/ml in Tris 0.1M pH 7.5) was added with a peristaltic pump (in 10 minutes) to reach a final ratio of 0.5 mg EDAC/mg PSC_{AH}. The resulting solution was left 110 min at +25° C. under stirring and pH regulation to obtain a final coupling time of 120 min. The solution was then neutralized by addition of 1 M Tris-Hcl pH 9.0 (1/10 of final volume) and left 30 minutes at +25° C. then overnight at +2° C. to +8° C.

The conjugate was clarified using a 10 μm filter and was purified using a Sephacryl S400HR column (Pharmacia, Sweden). The column was equilibrated in 10 mM Tris-HCl (pH 7.0), 0.075 M NaCl and the conjugate (approx. 460 mL) was loaded on the column (+2° C. to +8° C.). The elution pool was selected as a function of optical density at 280 nm. Collection started when absorbance increased to 0.05. Harvest continued until the Kd reached 0.20. The conjugate was filter sterilised at +20° C., then stored at +2° C. to +8° C. The resultant conjugate had a polysaccharide:protein ratio of 1:2-1:4 (w/w).

Various experiments adding EDAC over 10-45 minutes were carried out—in each case good quality MenC conjugates resulted. If, however the TT carrier was added last slowly to the MenC-ADH+EDAC mix this led to a gel—a conjugate that could not be purified.

Experiments were also carried out adding the EDAC all at once into the reaction but the final TT/PS ratio (2.7/1) (w/w) of the conjugate was lower than for the conjugate obtained via the reaction where EDAC was added over 10 minutes (3.3/1); furthermore the αTT and αPS antigenicity were both lower than that measured in respect of the conjugate made by the reaction where EDAC was added over 10 minutes.

### Note on Approximate % Derivatisation of the Polysaccharides

MenCAH: after CDAP treatment with ADH about 3.47% of hydroxyl groups were derivatized with ADH (with an estimation of two available hydroxyl groups per repeat subunit). For MenA: about 11.5% of hydroxyl groups derivatized with ADH (considering there is only one available hydroxyl group per repeat unit).

### Example 1b

### Preparation of Pneumococcal Capsular PS 3 Polysaccharide Conjugate

1) PS03-TT_{AH} process: PS03-TT_{AH}208

### Sizing by Emulsiflex

PS was weighed on the basis of 10% theoretical moisture content. The native PS was dissolved overnight in 2M NaCl at an initial concentration of 3 mg/ml. Before sizing, the solution of native PS was clarified on 5 μm cut-off filter.

A homogenizer EMULSIFLEX C-50 apparatus was used to reduce the molecular weight and the viscosity of the polysaccharide before the activation step. The efficiency of the sizing depends on the circuit pressure, the plunger alimentation pressure and on the total cycles number. In order to improve the efficiency of sizing (and consequently reduce the total number of cycles), the homogenizing cell of Emulsiflex was replaced with a cell with a fixed geometry (Microfluidics F20Y-0.75 μm interaction chamber). The aim of the sizing was to reduce the molecular weight and the viscosity of the PS without a critical decrease of its antigenicity.

The size reduction was done at 6000±500 psi and followed in-process by a measure of viscosity. The sizing was stopped when the target of 2.0±0.2 cp was reached.

### Filtration of Sized PS on 0.22 μm

Sized PS was filtered on a Millipak 40 membrane (cut-off 0.22 mm) at a flow-rate of 10 ml/min.

### TT Derivatization

The derivatization step was performed at 25° C. under continuous stirring in a T° controlled waterbath. TT was diluted in NaCl 0.2M to obtain a final TT concentration of 25 mg/ml. ADH was added in solid form to the TT solution to reach a 0.2M final concentration. After complete ADH dissolution, the solution was set at pH 6.2+/−0.1 with HCl.

EDAC was then added to the TT/ADH solution to reach a final 0.02M concentration. The pH was set at 6.2+/−0.1 with HCl and was kept under pH regulation during 1 hour.

After the derivatization step, the pH was raised up to pH9.5 with NaOH to stop the reaction. The solution was left during 2 hours under pH regulation before the diafiltration step.

### Diafiltration

TT_{AH} derivative was diafiltered in order to remove unreacted ADH and EDAC by-products. The diafiltration was performed on a centramate membrane (0.09 m², 10 kDa cut-off). The solution was dialysed against 20 volumes of 0.2M NaCl.

The follow-up of the diafiltration step was performed by a quantification of ADH (TNBS assay) in the permeate after 5, 10, 15 and 20 volumes of diafiltration.

### Filtration on 0.22 μm

TT_{AH} was finally filtered on 0.22 μm cut-off membrane (Millipack 40) at a flow-rate of 10 ml/min. The filtered TT_{AH} was then stored at −70° C.

### PS3-TT_{AH} Conjugate

The conditions of process were the following:

An initial PS3 concentration of 2 mg/ml in 2 M NaCl, an initial TT_{AH}/PS3 ratio of 1.5/1 (w/w), an EDAC concentration of 0.5 mg/mg PS, and a TT concentration of 10 mg/ml in 0.15M NaCl.

50 mg of PS3 were diluted in 2M NaCl to obtain a final PS concentration of 2 mg/ml. The purified TT_{AH} solution was diluted in 0.2M NaCl to reach a concentration of 10 mg/ml.

The PS3 solution was adjusted to pH5 with HCl.

EDAC was added in solid form to the PS3 solution in order to reach a final concentration of 0.5 mg EDAC/mg PS. The pH was adjusted to 5.0±0.05 with HCl and TT_{AH} was manually added in 11 minutes (aliquots/min). The resulting solution was incubated 109 min at +25° C. with stirring and pH regulation to obtain a final coupling time of 120 min. Then the solution was neutralized by addition of 1M Tris-HCl pH 7.5 and left 30 min at +25° C. The conjugate was finally clarified on a 5 μm membrane and injected on a Sephacryl S400HR column.

### 2) PS03-TT_{AH} Process: PS03_{AH}-TT215

### Sizing by Emulsiflex

As above.

Filtration of sized PS on 0.22 μm

As above.

### PS3 Derivatization

The derivatization step was performed at 25° C. under continuous stirring in a T° controlled waterbath. PS3 was diluted in NaCl 2M to obtain a final PS concentration of 3 mg/ml. The PS solution was set at pH6.0 before the addition of CDAP (0.25 mg/mg PS, dissolution at 100 mg/ml in a mix of acetonitrile/WFI). The pH was increased to pH9.5 with NaOH before the addition of ADH (8.9 mg ADH/mg PS, dissolution at 100 mg/ml in 0.2M NaCl). The pH was kept at 9.5 and regulated during 60 minutes. The percentage of derivatization corresponded to 2.4% (2.4 mg ADH/100 mg PS). This can be measured with known techniques: TNBS for the estimating ADH; and DMAB or resorcinol (Monsigny et al (1988) Anal. Biochem. 175, 525-530) for the PS quantification. In this case, TNBS dosage was 228 μg/ml and PS dosage: 5250 μg/ml.

Given the Mw of ADH is 174.2, and the Mw of the repeat unit of PS3 is 338.27 (having 1 COOH and 4 OH groups), there is 1.3 μmoles of ADH/15.52 μmole of repeat unit, or 1.3 μmoles of ADH/62.08 μmole of reactive hydroxyl group. 2.09% of PS3 hydroxyl groups were ADH modified hydroxyl groups.

### Diafiltration

PS3_{AH} derivative was diafiltered in order to remove unreacted ADH and CDAP by-products. The diafiltration was performed on a UFP-30-C-H24LA membrane (42 cm², 30 kDa cut-off). The solution was dialysed against 20 volumes of 0.2M NaCl.

The follow-up of the diafiltration step was performed by a quantification of ADH (TNBS assay) in the permeate after 5, 10, 15 and 20 volumes of diafiltration.

### Filtration on 0.22 μm

PS_{AH} was finally filtered on 0.22 μm cut-off membrane (Millipack 40) at a flow-rate of 10 ml/min. The filtered PS3_{AH} was then stored at 4° C.

### PS3_{AH}-TT Conjugate

The conditions of the process were the following:

An initial PS3 concentration of 2 mg/ml in 2 M NaCl, an initial TT/PS3_{AH} ratio of 1.5/1 (w/w), an EDAC concentration of 0.5 mg/mg PS, and a TT concentration of 10 mg/ml in 0.15M NaCl.

50 mg of PS3_{AH} was diluted in 0.2M NaCl to obtain a final PS concentration of 2 mg/ml. The purified TT solution was diluted in 0.2M NaCl to reach a concentration of 10 mg/ml. The PS3_{AH} solution was adjusted to pH5 with HCl.

EDAC was added in solid form to the PS3_{AH} solution in order to reach a final concentration of 0.5 mg EDAC/mg PS. The pH was adjusted to 5.0±0.05 with HCl and TT was added in 10 minutes using a peristaltic pump. The resulting solution was incubated 110 min at +25° C. with stirring and pH regulation to obtain a final coupling time of 120 min. Then the solution was neutralized by addition of 1M Tris-HCl pH 7.5 and left 30 min at +25° C. The conjugate was finally clarified on a 5 μm membrane and injected on a Sephacryl S400HR column.

3) PS03_{AH}-TT process: PS3_{AH}-TT217

### Sizing by Emulsiflex

As above.

### Filtration of Sized PS on 0.22 μm

As above.

### PS3 Derivatization

As for 215 conjugate.

### Diafiltration

As for 215 conjugate.

### Filtration on 0.22 μm

As for 215 conjugate.

### PS3_{AH}-TT Conjugate

The conditions of the process were the following:

An initial PS3 concentration of 2 mg/ml in 2 M NaCl, an initial TT/PS3_{AH} ratio of 1.5/1 (w/w), an EDAC concentration of 0.5 mg/mg PS, and a TT concentration of 10 mg/ml in 0.15M NaCl.

50 mg of PS3_{AH} was diluted in 0.2M NaCl to obtain a final PS concentration of 2 mg/ml. The purified TT solution was diluted in 0.2M NaCl to reach a concentration of 10 mg/ml. The PS3_{AH} and TT solutions were mixed and adjusted to pH5 with HCl.

EDAC was dissolved in a Tris 1M pH7.5 buffer. 40 μl of EDAC were added each minute (10 minutes to reach the EDAC/PS ratio (0.5 mg EDAC/mg PS)). The resulting solution was incubated 110 min at +25° C. under stirring and pH regulation to obtain a final coupling time of 120 min. Then the solution was neutralized by addition of 1M Tris-HCl pH 7.5 and left 30 min at +25° C. The conjugate was finally clarified on a 5 μm membrane and injected on a Sephacryl S400HR column.

### 4) PS3_{AH}-TT Process: PS3_{AH}-TT218

### Sizing by Emulsiflex

As above.

### Filtration of Sized PS on 0.22 μm

As above.

### PS3 Derivatization

The derivatization step was performed at 25° C. with continuous stirring in a T° controlled waterbath. PS3 was diluted in NaCl 2M to obtain a final PS concentration of 3 mg/ml. EDAC was added in solid form to reach an EDAC/PS ratio of 0.1 mg/mg PS. After complete dissolution, the pH of the solution was set at 5. ADH (8.9 mg ADH/mg PS, dissolution at 100 mg/ml in 0.2M NaCl) was then added using a peristaltic pump in 44 minutes (though as such an excess of ADH was present, direct addition would also have been OK). The pH was kept at 5.0+/−0.1 and regulated during 120 minutes (44′+76′). The reaction was stopped by increasing the pH to 7.5 using sodium hydroxide. The percentage of derivatization corresponded to 3.7% (mg ADH/mg PS). TNBS dosage was 220 μg/ml and PS dosage was 5902 μg/ml, thus there is 1.26 μmoles of ADH/17.44 μmole of repeat unit (=μmole of reactive COOH group). Thus, 7.22% of PS3 carboxyl groups were ADH modified COOH groups.

### Diafiltration

PS3_{AH} derivative was diafiltered in order to remove unreacted ADH and EDAC by-products. The diafiltration was performed on a UFP-30-C-H24LA membrane (42 cm², 30 kDa cut-off). The solution was dialysed against 23 volumes of 0.2M NaCl.

The follow-up of the diafiltration step was performed by a quantification of ADH (TNBS assay) in the permeate after 5, 10, 15 and 20 volumes of diafiltration

### Filtration on 0.22 μm

PS_{AH} was finally filtered on 0.22 μm cut-off membrane (Millipack 40) at a flow-rate of 10 ml/min. The filtered PS3_{AH} was then stored at 4° C.

### PS3_{AH}-TT Conjugate

The conditions of the process were the following:

An initial PS3_{AH} concentration of 2 mg/ml in 2 M NaCl, an initial TT/PS3_{AH} ratio of 1.5/1 (w/w), an EDAC concentration of 0.5 mg/mg PS, and a TT concentration of 10 mg/ml in 0.15M NaCl.

50 mg of PS3_{AH} was diluted in 0.2M NaCl to obtain a final PS concentration of 2 mg/ml. The purified TT solution was diluted in 0.2M NaCl to reach a concentration of 10 mg/ml. The PS3_{AH} and TT solutions were mixed together.

The pH was adjusted to 5.0±0.05 with HCl and EDAC was manually added in 10 minutes (equal part-aliquots added each minute). The resulting solution was incubated 110 min at +25° C. with stirring and pH regulation to obtain a final coupling time of 120 min. Then the solution was neutralized by addition of 1M Tris-HCl pH 7.5 and left 30 min at +25° C. The conjugate was finally clarified on a 5 μm membrane and injected on a Sephacryl S400HR column.

### CONCLUSIONS

Different conjugates were made using carbodiimide chemistry in the conjugation step. The last component added in the reaction solution can be either the TT protein or the EDAC reagent. The time of addition can have an effect on the resulting conjugates.

### PS3_{AH}TT215 & 217 Conjugates:

The same components and conditions were used to prepare both conjugates. The way in which the last component was added was different. PS3_{AH}TT217 conjugate led to a product which met in-vitro criteria. This one was made by adding EDAC in 10 minutes. PS3_{AH}TT215 conjugate, however, could not be filtered on sterile membrane. For this one, the last component added in the reaction medium was the TT (in 10 minutes).

Final TT/PS ratios were highly different for both conjugates. (0.98/1 vs 0.50/1). If EDAC is added first to the PS_{AH} (having both reactive amino and carboxyl groups) this can lead to intra cross-linking of hydrazine and carboxylic groups present on the polysaccharide, and thus could lead to a more cross-linked conjugate with a weaker final ratio after the addition of TT in 10 minutes.

This effect is not observed for the PS3_{AH}TT217 conjugate. The TT incorporation worked better by the addition of EDAC in 10 minutes, perhaps due to lower intra cross-linking, and better inter cross-linking between hydrazine groups of the PS3_{AH} and carboxylic groups of the protein.

In the case of the 218 conjugate, as the PS3 EDAC derivatisation only partially derivatises the polysaccharide (to keep the majority of the polysaccharides epitopes intact), again both reactive amino and carboxyl groups are present, hence why slow addition of EDAC in a final conjugation step is also beneficial.

Slow TT addition in the final conjugation step was beneficial (however) for the 208 conjugate where the TT was ADH derivatised (and comprises amino and carboxyl groups), whereas the PS3 was left with its native reactive —OH and —COOH groups as part of its repeating subunit. The addition of EDAC to PS 3 did not have the above cross-linking effect, and the slow addition of the derivatised TT yielded conjugate with good in vitro characteristics—see below.

### In-Vitro Characterization:

| | Derivatization/ | | Final component |
|---|---|---|---|
| Conj. | Chemistry | Conjugation/Chemistry | addition |
| 208 | TT/ADH → EDAC | PS-TT_{AH} → EDAC | TT_{AH} added in |
| | | | 11 minutes |
| 215 | PS3/ADH → CDAP | PS_{AH}-TT → EDAC | TT added in 10 |
| | | | minutes |
| 217 | PS3/ADH → CDAP | PS_{AH}-TT → EDAC | EDAC added in |
| | | | 10 minutes |
| 218 | PS3/ADH → EDAC | PS_{AH}-TT → EDAC | EDAC added in |
| | | | 10 minutes |

| | | [PS] | [TT] | In. TT/PS | [EDAC] | Coupl. time |
|---|---|---|---|---|---|---|
| Conj. | PS | (mg/ml) | (mg/ml) | ratio (w/w) | (mg/mg PS) | (min) |
| 208 | C6E02 | 2.0 | 10 | 1.5/1 | 0.5/1 | 120 |
| | | | (TT_{AH}), pump | | | |
| 215 | 3_{AH}001 | 2.0 | 10 | 1.5/1 | 0.5/1 | 120 |
| | (CDAP) | | pump | | | |
| 217 | 3_{AH}001 | 2.0 | 10 | 1.5/1 | 0.5/1 | 120 |
| | (CDAP) | | | | (Fractions) | |
| 218 | 3_{AH}002 | 2.0 | 10 | 1.5/1 | 0.5/1 | 120 |
| | (EDAC) | | | | (Fractions) | |

| | F. TT/PS | Yield | Filtr. | Free | αPS/αPS | αTT/αPS |
|---|---|---|---|---|---|---|
| | ratio | PS | yield | PS | (%) | (%) |
| Conj. | (w/w) | rec (%) | rec (%) | (%) | Antigenicity | Antigenicity |
| 208 | 1.84/1 | 69 | 95 | 10.2 | 99 | 103 |
| | | | | | | 100* |
| 215 | 0.50/1 | 17 | 27 | — | — | — |
| 217 | 0.98/1 | 66 | 100 | 0.7 | 17 | 103 |
| | | | | | | 100* |
| 218 | 0.88/1 | 74 | 101 | 11.0 | 34 | 222 |
| | | | | | | 216* |

*relative to the 208 conjugate

### Example 1c

### Preparation of S. typhi Vi Polysaccharide Conjugate of the Invention

### Sizing by Emulsiflex

PS was weighed on the basis of 15% theoretical moisture content. The native PS is dissolved overnight in WFI at an initial concentration of 7 mg/ml. Before the sizing, the solution of native PS is clarified on 10 μm cut-off filter at a flow-rate of 50 ml/min.

A homogenizer EMULSIFLEX C-50 apparatus was used to reduce the molecular weight and the viscosity of the polysaccharide before the activation step. The efficiency of the sizing depends on the circuit pressure, the plunger alimentation pressure and on the total cycles number. In order to improve the efficiency of sizing (and consequently reduce the total number of cycles), the homogenizing cell of Emulsiflex was replaced by a cell with a fixed geometry (Microfluidics F20Y-0.75 μm interaction chamber). The aim of the sizing is to reduce the molecular weight and the viscosity of the PS without a critical decrease of its antigenicity.

The size reduction was realized at 15000±500 psi and followed in-process by a measure of viscosity. The sizing is stopped when the target of 5.0±0.3 cp is reached.

### Filtration of Sized PS on 0.22 μm

Sized PS is filtered on a Millipak 40 membrane (cut-off 0.22 mm) at a flow-rate of 10 ml/min. The filtered sized PS is stored at −20° C.

### Derivatization of Polysaccharide Vi

1.5 g of sized Vi PS was dissolved at 25° C. in EPI under agitation (5 mg/ml). 13.35 g of ADH (8.9 mg ADH/mg PS) is added to the PS solution. After complete dissolution pH was adjusted at pH 5.0±0.05 with 1N HCl. EDAC (0.1 mg/mg PS) was added in a solid form. The solution was left 60 min at 25° C. Then the solution was neutralized by addition of 1M Tris-HCl pH 7.5 and left at least 30 min at 25° C. (maximum 2 hours). The level of derivatization was estimated to be 4.55% using the TNBS dosage (mg ADH/100 mg PS). TNBS dosage was 200 μg/ml and PS dosage was 4034 μg/ml; thus 0.0697 μmoles of ADH/16.46 μmole of repeat unit (Mw 245). 1.3 μmoles of ADH/16.46 μmole of reactive COOH group on Vi, thus 7% of Vi COOH groups were ADH modified COOH groups.

### Diafiltration

PSVi_{AH} derivative was diafiltered in order to remove unreacted ADH and EDAC by-products. The diafiltration was performed on a centramate membrane (0.09 m², 10 kDa cut-off). The solution was dialysed against 20 volumes of 0.2M NaCl.

The follow-up of the diafiltration step was performed by a quantification of ADH (TNBS assay) in the permeate after 3, 5, 10 and 20 volumes of diafiltration

### Filtration on 0.22 μm

PSVi_{AH} was finally filtered on 0.22 μm cut-off membrane (Millipack 40) at a flow-rate of 10 ml/min. The filtered PSViAH was stored at +2/+8° C. for a maximum of 4 days.

### PSVi_{AH}-TT Conjugates

The conditions of process were the following:

An initial PSViAH concentration of 2 mg/ml in 0.2 M NaCl, an initial TT/PSViAH ratio of 2.5/1 (w/w), an EDAC concentration of 0.25 mg/mg PS and a TT concentration of 10 mg/ml in 0.2M NaCl.

1 g of PSVi_{AH} was diluted in 0.2M NaCl to obtain a final PS concentration of 2 mg/ml (uronic acid dosage). The purified TT solution was diluted in 0.2M NaCl to reach a concentration of 10 mg/ml.

TT was added to the PSVi_{AH} solution in order to reach a final ratio of 2.5 mg TT/mg PS. The pH is adjusted to 5.0±0.05 with 1N HCl. The EDAC solution (7.5 mg/ml in 0.1M Tris pH 7.5) was then added (in 10 minutes with a peristaltic pump) to reach 0.25 mg EDAC/mg PSVi_{AH}. The resulting solution was incubated 50 min at +25° C. with stirring and pH regulation to obtain a final coupling time of 60 min. Then the solution was neutralized by addition of 1M Tris-HCl pH 7.5 and left 30 min at +25° C. The conjugate was transferred at 4° C. and is left overnight under continuous slow stirring before the chromatography step.

### Purification

Prior to the elution on Sephacryl S400HR, the conjugate was clarified using a 10 μm Kleenpak filter. The flow rate was fixed at 100 ml/min. The conjugate was then injected on Sephacryl S400HR and the collection pool was based on a Kd value. The following criterion was used for the pool collection: from OD=0.05 at 280 nm harvesting started, and finished when Kd=0.22.

### Sterilizing Filtration

Before filtration, the bulk was brought back to room temperature. Then the conjugate was filtered on an Opticap 4″ sterilizing membrane. The flow rate was fixed at 30 ml/min.

### Analytical

The resulting conjugate had a final TT/PS ratio (w/w) of 2.44/1, a free PS content of 3.7% and a αPS/αPS antigenicity of 58%.

### Example 1c(ii)

### Other Means to Prepare of S. typhi Vi Polysaccharide Conjugates of the Invention

It is envisioned that conjugation may be carried out with other conditions to those described in Example 1c. In short, Example 1c is carried out, but the following conditions are altered:

| | Derivatization of Vi - | | Coupling with TT - | | | |
|---|---|---|---|---|---|---|
| | quantity of EDAC | | quantity of EDAC | | Total TT Coupling | |
| Conjugation | added | | added | | time | |
| 1 (conditions of | 0.1 | mg/mg Vi PS | 0.25 | mg/mg Vi_{AH} PS | 60 | min |
| Example 1c) | | | | | | |
| 2 | 0.1 | mg/mg Vi PS | 0.25 | mg/mg Vi_{AH} PS | 20, 30, 40 | min |
| 3 | 0.1 | mg/mg Vi PS | 0.2 | mg/mg Vi_{AH} PS | 60 ± 30 | min |
| 4 | 0.1 | mg/mg Vi PS | 0.15 | mg/mg Vi_{AH} PS | 60 ± 30 | min |
| 5 | 0.1 | mg/mg Vi PS | 0.1 | mg/mg Vi_{AH} PS | 60 ± 30 | min |
| 6 | 0.05 | mg/mg Vi PS | 0.25 | mg/mg Vi_{AH} PS | 60 or 20, 30, 40 | min |
| 7 | 0.05 | mg/mg Vi PS | 0.2 | mg/mg Vi_{AH} PS | 60 ± 30 | min |
| 8 | 0.05 | mg/mg Vi PS | 0.15 | mg/mg Vi_{AH} PS | 60 ± 30 | min |
| 9 | 0.05 | mg/mg Vi PS | 0.10 | mg/mg Vi_{AH} PS | 60 ± 30 | min |
| 10 | 0.025 | mg/mg Vi PS | 0.25 | mg/mg Vi_{AH} PS | 60 ± 30 | min |
| 11 | 0.025 | mg/mg Vi PS | 0.2 | mg/mg Vi_{AH} PS | 60 ± 30 | min |
| 12 | 0.025 | mg/mg Vi PS | 0.15 | mg/mg Vi_{AH} PS | 60 ± 30 | min |
| 13 | 0.025 | mg/mg Vi PS | 0.1 | mg/mg Vi_{AH} PS | 60 ± 30 | min |

### Example 1d

### Preparation of Other Polysaccharide Conjugates

The covalent binding of Haemophilus influenzae (Hib) PRP polysaccharide to TT was carried out by a coupling chemistry developed by Chu et al (Infection and Immunity 1983, 40 (1); 245-256). Hib PRP polysaccharide was activated by adding CNBr and incubating at pH10.5 for 6 minutes. The pH was lowered to pH8.75 and adipic acid dihydrazide (ADH) was added and incubation continued for a further 90 minutes. The activated PRP was coupled to purified tetanus toxoid via carbodiimide condensation using 1-ethyl-3-(3-dimethyl-aminopropyl)carbodiimide (EDAC). EDAC was added to the activated PRP to reach a final ratio of 0.6 mg EDAC/mg activated PRP. The pH was adjusted to 5.0 and purified tetanus toxoid was added to reach 2 mg TT/mg activated PRP. The resulting solution was left for three days with mild stirring. After filtration through a 0.45 μm membrane, the conjugate was purified on a sephacryl S500HR (Pharmacia, Sweden) column equilibrated in 0.2M NaCl.

MenC-TT conjugates were produced using native polysaccharides (of over 150 kDa as measured by MALLS) or were slightly microfluidised. MenA-TT conjugates were produced using either native polysaccharide or slightly microfluidised polysaccharide of over 60 kDa as measured by the MALLS method of example 2. MenW and MenY-TT conjugates were produced using sized polysaccharides of around 100-200 kDa as measured by MALLS (see example 2). Sizing was by microfluidisation using a homogenizer Emulsiflex C-50 apparatus. The polysaccharides were then filtered through a 0.2 μm filter.

Activation and direct coupling were performed as described in WO96/29094 and WO 00/56360. Briefly, the polysaccharide at a concentration of 10-20 mg/ml in 2M NaCl pH 5.5-6.0 was mixed with CDAPsolution (100 mg/ml freshly prepared in acetonitrile/WFI, 50/50) to a final CDAP/polysaccharide ratio of 0.75/1 or 1.5/1. After 1.5 minutes, the pH was raised with sodium hydroxide to pH10.0. After three minutes tetanus toxoid was added to reach a protein/polysaccharide ratio of 1.5/1 for MenW, 1.2/1 for MenY, 1.5/1 for MenA or 1.5/1 for MenC. The reaction continued for one to two hours.

After the coupling step, glycine was added to a final ratio of glycine/PS (w/w) of 7.5/1 and the pH was adjusted to pH9.0. The mixture was left for 30 minutes. The conjugate was clarified using a 10 μm Kleenpak filter and was then loaded onto a Sephacryl S400HR column using an elution buffer of 150 mM NaCl, 10 mM or 5 mM Tris pH7.5. Clinical lots were filtered on an Opticap 4 sterilizing membrane. The resultant conjugates had an average polysaccharide:protein ratio of 1:1-1:5 (w/w).

### Example 2

### Determination of Molecular Weight Using MALLS

Detectors were coupled to a HPLC size exclusion column from which the samples were eluted. On one hand, the laser light scattering detector measured the light intensities scattered at 16 angles by the macromolecular solution and on the other hand, an interferometric refractometer placed on-line allowed the determination of the quantity of sample eluted. From these intensities, the size and shape of the macromolecules in solution can be determined.

The mean molecular weight in weight (M_{w}) is defined as the sum of the weights of all the species multiplied by their respective molecular weight and divided by the sum of weights of all the species.
a) Weight-average molecular weight: -Mw-

$${M}_{w} = \frac{{\sum }^{ } {W}_{i} . {M}_{i}}{{\sum }^{ } {W}_{i}} = \frac{{m}_{2}}{{m}_{1}}$$

b) Number-average molecular weight: -Mn-

$${M}_{n} = \frac{{\sum }^{ } {N}_{i} . {M}_{i}}{{\sum }^{ } {N}_{i}} = \frac{{m}_{1}}{{m}_{0}}$$

c) Root mean square radius: -Rw- and R²w is the square radius defined by:

$${R}^{2} w or ( {r}^{2} ) w = \frac{{\sum }^{ } {m}_{i} . {r}_{i}^{2}}{{\sum }^{ } {m}_{i}}$$

(-mᵢ- is the mass of a scattering centre i and -rᵢ- is the distance between the
scattering centre i and the center of gravity of the macromolecule).
d) The polydispersity is defined as the ratio -Mw/Mn-.

Meningococcal polysaccharides were analysed by MALLS by loading onto two HPLC columns (TSKG6000 and 5000PWxl) used in combination. 25 μl of the polysaccharide were loaded onto the column and was eluted with 0.75 ml of filtered water. The polysaccharides are detected using a light scattering detector (Wyatt Dawn DSP equipped with a 10 mW argon laser at 488 nm) and an inferometric refractometer (Wyatt Otilab DSP equipped with a P100 cell and a red filter at 498 nm).

The molecular weight polydispersities and recoveries of all samples were calculated by the Debye method using a polynomial fit order of 1 in the Astra 4.72 software.

### Example 3

### Clinical Trial Assessing the Effect of a Linker in MenA in a MenACWY Conjugate Vaccine

A single dose of different formulations of MenACWY vaccine was administered to teenagers of 15-19 years in 5 groups of 25 subjects in a 1:1:1:1:1 randomised trial. The formulations tested were:

F1—MenACWY conjugated to tetanus toxoid with the MenA conjugate containing an AH (ADH) spacer (made according to example 1)—5/5/5/5 μg
F2—MenACWY conjugated to tetanus toxoid with the MenA conjugate containing an AH spacer (made according to example 1)—2.5/5/2.5/2.5 μg
F3—MenACWY conjugated to tetanus toxoid with the MenA conjugate containing an AH spacer (made according to example 1)—5/5/2.5/2.5 μg
F4—MenACWY conjugated to tetanus toxoid with no spacer in any conjugate—5/5/5/5 μg
Control group—Mencevax™ ACWY

On day 30 after inoculation, a blood sample was taken from the patients.

The blood samples were used to assess the percentage of SBA-MenA, SBA-MenC, SBA-MenW135 and SBA-MenY responders one month after the vaccine dose. A vaccine response was defined as 1) for initially seronegative subjects—a post-vaccination antibody titre ≧1/32 at 1 month or 2) for initially seropositive subjects—antibody titre of ≧4 fold the pre-vaccination antibody titre.

### Results

As shown in the Table below, the use of a spacer in the MenA conjugate led to an increased immune response against MenA. The percentage of responders rose from 66% to 90-95% when the AH spacer was added. This was reflected in an increase in SBA GMT from 4335 to 10000 and an increase in GMC from 5 to 20-40. Surprisingly, the use of a AH spacer also led to an increased immune response against MenC as seen by an increase in the percentage of responders and an increase in the SBA GMT. An increase could also be seen in the SBA-GMT against MenY (6742-7122) and against MenW (4621-5418) when a spacer was introduced.

| | % SBA MenA | SBA-MenA | Anti-PSA GMC |
|---|---|---|---|
| Formulation | responders | GMT | μg/ml ELISA |
| F1 5AH/5/5/5 | 90.9 | 9805 | 20.38 |
| F2 2.5AH/5/2.5/2.5 | 75 | 8517 | 29.5 |
| F3 5AH/5/2.5/2.5 | 95.5 | 10290 | 47.83 |
| F4 5/5/5/5 | 66.7 | 4335 | 5.46 |
| Mencevax ™ | 85.7 | 8022 | 27.39 |
| | % SBA MenC | SBA-MenC | Anti-PSC GMC |
| Formulation | responders | GMT | μg/ml ELISA |
| F1 5AH/5/5/5 | 69.6 | 3989 | 12.11 |
| F2 2.5AH/5/2.5/2.5 | 81.8 | 3524 | 12.78 |
| F3 5AH/5/2.5/2.5 | 81.8 | 3608 | 8.4 |
| F4 5/5/5/5 | 73.9 | 2391 | 8.84 |
| Mencevax ™ | 90.0 | 5447 | 38.71 |
| | % SBA MenW | SBA-MenW | Anti-PSW GMC |
| Formulation | responders | GMT | μg/ml ELISA |
| F1 5AH/5/5/5 | 95 | 5418 | 9.65 |
| F2 2.5AH/5/2.5/2.5 | 85 | 4469 | 14.55 |
| F3 5AH/5/2.5/2.5 | 95.5 | 4257 | 6.39 |
| F4 5/5/5/5 | 95.5 | 4621 | 10.7 |
| Mencevax ™ | 86.4 | 2714 | 13.57 |
| | % SBY MenY | SBA-MenY | Anti-PSY GMC |
| Formulation | responders | GMT | μg/ml ELISA |
| F1 5AH/5/5/5 | 91.3 | 7122 | 16.3 |
| F2 2.5AH/5/2.5/2.5 | 87.5 | 5755 | 12.52 |
| F3 5AH/5/2.5/2.5 | 80 | 5928 | 8.88 |
| F4 5/5/5/5 | 91.3 | 6742 | 13.88 |
| Mencevax ™ | 91.7 | 4854 | 21.02 |

### Example 4

### Clinical Trial Assessing the Effect of a Linker in MenA and MenC Conjugates in a MenACWY Conjugate Vaccine

A single dose of different formulations of MenACWY vaccine was administered to teenagers of 15-19 years in 5 groups of 25 subjects in a 1:1:1:1:1 randomised trial. The formulations tested were:

F1—MenACWY conjugated to tetanus toxoid with the MenA and MenC conjugates containing an AH spacer (made according to example 1)—2.5/2.5/2.5/2.5 μg
F2—MenACWY conjugated to tetanus toxoid with the MenA and MenC conjugates containing an AH spacer (made according to example 1)—5/5/2.5/2.5 μg
F3—MenACWY conjugated to tetanus toxoid with the MenA and MenC conjugates containing an AH spacer (made according to example 1)—5/5/5/5 μg
F4—MenACWY conjugated to tetanus toxoid with the MenA conjugate containing an AH spacer (made according to example 1)—5/5/5/5 μg
Control group—Mencevax™ ACWY

On day 30 after inoculation, a blood sample was taken from the patients.

The blood samples were used to assess the percentage of SBA-MenA, SBA-MenC, SBA-MenW135 and SBA-MenY responders one month after the vaccine dose. A vaccine response was defined as 1) for initially seronegative subjects—a post-vaccination antibody titre ≧1/32 at 1 month or 2) for initially seropositive subjects—antibody titre of ≧4 fold the pre-vaccination antibody titre.

### Results

The introduction of an AH spacer into the MenC conjugate led to an increase in the immune response against MenC as shown in the Table below. This is demonstrated by an increase in SBA GMT from 1943 to 4329 and an increase in anti-PSC GMC from 7.65 to 13.13. Good immune responses against MenA, MenW and MenY were maintained.

| | % SBA MenA | SBA-MenA | Anti-PSA GMC |
|---|---|---|---|
| Formulation | responders | GMT | μg/ml ELISA |
| F1 | 75 | 8417 | 20.23 |
| 2.5AH/2.5AH/2.5/2.5 | | | |
| F2 5AH/5AH/2.5/2.5 | 72 | 6299 | 16.07 |
| F3 5AH/5AH/5/5 | 87 | 9264 | 27.26 |
| F4 5AH/5/5/5 | 77.3 | 9632 | 20.39 |
| Mencevax ™ | 78.3 | 8284 | 12.93 |
| | % SBA MenC | SBA-MenC | Anti-PSC GMC |
| Formulation | responders | GMT | μg/ml ELISA |
| F1 | 88 | 3619 | 12.82 |
| 2.5AH/2.5AH/2.5/2.5 | | | |
| F2 5AH/5AH/2.5/2.5 | 88 | 2833 | 13.32 |
| F3 5AH/5AH/5/5 | 95.8 | 4329 | 13.13 |
| F4 5AH/5/5/5 | 95.8 | 1943 | 7.65 |
| Mencevax ™ | 91.7 | 1567 | 16.55 |
| | % SBA MenW | SBA-MenW | Anti-PSW GMC |
| Formulation | responders | GMT | μg/ml ELISA |
| F1 | 100 | 5656 | 7 |
| 2.5AH/2.5AH/2.5/2.5 | | | |
| F2 5AH/5AH/2.5/2.5 | 96 | 4679 | 5.4 |
| F3 5AH/5AH/5/5 | 91.3 | 4422 | 4.45 |
| F4 5AH/5/5/5 | 88 | 4947 | 7.67 |
| Mencevax ™ | 96 | 3486 | 11.93 |
| | % SBY MenY | SBA-MenY | Anti-PSY GMC |
| Formulation | responders | GMT | μg/ml ELISA |
| F1 | 75 | 3891 | 17.81 |
| 2.5AH/2.5AH/2.5/2.5 | | | |
| F2 5AH/5AH/2.5/2.5 | 92 | 3968 | 11.96 |
| F3 5AH/5AH/5/5 | 79.2 | 2756 | 9.51 |
| F4 5AH/5/5/5 | 80 | 3914 | 16.76 |
| Mencevax ™ | 88 | 3056 | 21.41 |

1.-49. (canceled)

50. An immunogenic composition or vaccine comprising a pharmaceutically acceptable excipient and a Vi capsular saccharide-protein carrier conjugate, said conjugate being obtainable by a method of conjugating a saccharide to a protein carrier using carbodiimide condensation chemistry, wherein the saccharide comprises or has been derivatised to comprise, amino and/or carboxyl groups, and wherein the protein carrier comprises, or has been derivatised to comprise, amino and/or carboxyl groups, comprising the steps of:
I)—if the protein carrier comprises both amino and carboxyl groups and the saccharide comprises either amino or carboxyl groups:
a) mixing the saccharide and aliquot of carbodiimide required to perform the conjugation, and
b) adding the aliquot of protein carrier required over a period of 35 seconds to 6 hours;
II)—if the saccharide comprises both amino and carboxyl groups and the protein carrier comprises either amino or carboxyl groups:
a) mixing the protein carrier and aliquot of carbodiimide required to perform the conjugation, and
b) adding the aliquot of saccharide required over a period of 35 seconds to 6 hours;
III)—if the saccharide comprises both amino and carboxyl groups and the protein carrier comprises both amino and carboxyl groups:
a) mixing the protein carrier and saccharide, and
b) adding the aliquot of carbodiimide required to perform the conjugation over a period of 35 seconds to 6 hours.

51. The immunogenic composition or vaccine of claim 50 wherein the Vi saccharide-protein carrier conjugate comprises 0.5-15, 1-10, 2.0-7.5 or 2.5-5 μg of Vi saccharide per human dose.

52. The immunogenic composition or vaccine of claim 50 further comprising a Hib capsular saccharide-protein carrier conjugate.

53.-68. (canceled)

69. The immunogenic composition or vaccine of claim 50 further comprising a DTP (DTPa or DTPw) vaccine.

70. (canceled)

71. The immunogenic composition or vaccine of claim 50 further comprising a Hepatitis B vaccine wherein the Hepatitis B antigen is hepatitis B surface antigen.

72. (canceled)

73. The immunogenic composition or vaccine of claim 50 further comprising a Hepatitis A vaccine.

74. (canceled)

75. The immunogenic composition or vaccine of claim 50 further comprising a Polio virus vaccine.

76. (canceled)

77. The immunogenic composition or vaccine of claim 50 further comprising one or more meningococcal capsular saccharide—protein carrier conjugates where the capsular saccharide(s) are derived from the following meningococcal serogroups: A, C, W135, Y, A and C, A and W135, A and Y, C and
W135, C and Y, W135 and Y, A and C and W135, A and C and Y, A and W135 and Y, C and W135 and Y, A and C and W135 and Y.

78. The immunogenic composition or vaccine of claim 50 further comprising a malaria vaccine.

79. The immunogenic composition or vaccine of claim 78, wherein the malaria vaccine is RTS,S.

80. The immunogenic composition or vaccine of claim 52 wherein the Vi and Hib capsular saccharide conjugates are co-lyophilised.

81.-88. (canceled)

89. A method of making the immunogenic composition or vaccine of claim 50 comprising the steps of conjugating a Vi capsular saccharide by the method of claim 50 and formulating the resulting saccharide-protein carrier conjugate with a pharmaceutically acceptable excipient.

90. A vaccine kit for concomitant or sequential administration comprising two multi-valent immunogenic compositions for conferring protection in a host against disease caused by Bordetella pertussis, Clostridium tetani, Corynebacterium diphtheriae, Salmonella typhi and Haemophilus influenzae, said kit comprising a first container comprising:
tetanus toxoid (TT),
diphtheria toxoid (DT), and
whole cell or acellular pertussis components (Pw or Pa);
and a second container comprising the immunogenic composition or vaccine of claim 52.

91.-93. (canceled)

94. Method of preventing or treating disease comprising the step of administering an effective dose of the immunogenic composition or vaccine of claim 50 to a patient in need thereof.

95. (canceled)
